(19) United States
(12) Patent Application Publication
(10) Pub. No.: US 2015/0291515 A1
(43) Pub. Date: Oct. 15, 2015

(54) SALTS CONTAINING TRIHYDROPERFLUOROALKOXYBUTANESULFONATE OR TRIHYDROPERFLUOROALKOXYPROPANESULFONATE ANION

(71) Applicant: MERCK PATENT GMBH, Darmstadt (DE)

(72) Inventors: Marc Uerdingen, Lohmar (DE); Roy Van Hal, Koeln (DE); Natalie Wehrum, Melsbach (DE); Susanne Reute, Langenfeld (DE); Oliver Stranowsky, Darmstadt (DE)

(73) Assignee: MERCK PATENT GMBH, Darmstadt (DE)

(21) Appl. No.: 14/439,402

(22) PCT Filed: Sep. 30, 2013
(86) PCT No.: PCT/EP2013/002932

§ 371 Date: Apr. 29, 2015

(30) Foreign Application Priority Data
Oct. 31, 2012 (DE) 10 2012 021 452.9

(51) Int. Cl. C07C 309/10; C10M 105/72

(52) U.S. Cl. CPC C07C 309/10; C10M 105/72

(57) ABSTRACT

The invention relates to compounds containing trihydroperfluoroalkoxybutanesulfonate or trihydroperfluoroalkoxypropane sulfonate anions, processes preparation and use thereof, in particular as anticorrosion additives together with ionic liquids.

The invention relates to salts containing trihydroperluoroalkoxybutanesulfonate or trihydroperfluoroalkoxypropanesulfonate anion, processes for the preparation and use thereof, in particular as anticorrosion additives together with ionic liquids which are not phosphates or alkylsulfates.

Most conventional anticorrosion additives are designed for hydrophobic base oils, for example mineral oils or ester oils, and therefore have only low miscibility with ionic compounds.

However, ionic compounds, in particular ionic liquids, are increasingly being described and researched as constituent of lubricant compositions or base oils for lubricant compositions or lubricating grease compositions. The novel lubricants based on ionic compounds, in particular based on ionic liquids, are used, for example, in vehicle technology, conveying technology, machine construction, office technology, in industrial plants and machines, in household machines and entertainment electronics.

An ionic liquid is taken to mean salts which generally consist of an organic cation and an inorganic anion. They do not contain any neutral molecules and usually have melting points below 373 K [Wasserscheid P, Keim W, 2000, Angew. Chem. 112: 3926]. Due to their salt character, ionic liquids have unique substance properties, such as, for example, a low vapour pressure, a liquid state over a broad temperature range, are non-flammable, exhibit high electrical conductivity, high electrochemical and thermal stability and good tribological properties.

The object of the invention is therefore to identify ionic compounds which reduce the tendency of ionic compounds to corrode metals or metal alloys and are infinitely miscible therewith.

This problem is solved in accordance with the invention by the subject-matters of the independent claims. Advantageous embodiments are the subject-matter of the dependent claims.

It has been found, surprisingly, that the compounds of the formula I, as described below, are able to considerably reduce the corrosion tendency of ionic compounds, in particular of ionic liquids, which are not phosphates or alkylsulfates.

The invention accordingly relates to compounds of the formula I,

[Kt]^{z+}z[SO₃—(CH₂)ₓ—O—CH₂—(CF₂—CF₂)_{y}—H]⁻ I

in which [Kt]^{z+} denotes an inorganic or organic cation, z denotes 1, 2, 3 or 4, y denotes 1, 2 or 3 and x denotes 3 or 4, where the compounds sodium nonafluoropropoxypropanesulfonate, sodium octafluoropentoxypropanesulfonate and sodium dodecafluoroheptoxypropanesulflonate are excluded.

The sodium compounds excluded above are known from Georg Sonnek et al, Journal of Organometallic Chemistry 1991, 405, 179-182, oligosiloxanes containing functional groups.

Preferred compounds of the formula I are compounds containing an organic cation.

The variable x is 3 or 4, preferably 4.

The variable y is 1, 2 or 3, preferably 2.

Particularly preferred compounds of the formula I are compounds containing an organic cation, as preferably or particularly preferably described below, where y denotes 2 and x denotes 3 or 4.

Very particularly preferred compounds of the formula I are compounds containing an organic cation, as preferably or particularly preferably described below, where y denotes 2 and x denotes 4.

Preferred organic cations of the formula I are selected from

sulfonium cations of the formula (1))

[(R^{o}₃S]⁺ (1),

where R^{o} in each case, independently of one another, denotes (R′″)₂N,
a linear or branched alkyl group having 1 to 8 C atoms,
an unsubstituted phenyl group or a phenyl group which is mono- or polysubstituted by R¹*, OR′, SR′, N(R′)₂, CN or halogen,
where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl,
R¹* in each case, independently of one another, denotes unfluorinated or partially fluorinated linear or branched alkyl group having 1 to 8 C atoms, saturated cycloalkyl group having 3 to 7 C atoms or unsubstituted or substituted phenyl and
R′″ in each case, independently of one another, denotes a linear or branched alkyl group having 1 to 6 C atoms;
ammonium cations of the formula (2)

[NR₄]⁺ (2),

where
R in each case, independently of one another,
denotes H, OR′ or N(R′)₂, with the assumption that a maximum of one substituent R in formula (3) denotes OR′ or N(R′)₂,
denotes a linear or branched alkyl group having 1 to 20 C atoms,
denotes a linear or branched alkenyl group having 2 to 20 C atoms and at least one double bond,
denotes a linear or branched alkynyl group having 2 to 20 C atoms and at least one triple bond or
denotes a saturated, partially unsaturated, unsaturated or aromatic cyclic group having 3 to 7 C atoms, which may be mono- or polysubstituted by a linear or branched alkyl group having 1 to 6 C atoms,
where one or two R may be partially replaced by halogen, preferably —F and/or —Cl, and/or by —OR′, —CN, —N(R′)₂, —C(O)OR′, —C(O)R′, —C(O)N(R′)₂, —SO₂N(R′)₂, —C(O)X, —SR′, —S(O)R′, —SO₂R′ and where one or two carbon atoms of the radical R which are not adjacent and are not in the α-position may be replaced by atoms and/or atom groups selected from the group —O—, —S—, —S(O)—, —SO₂—, —SO₂O—, —C(O)—, —C(O)O—, —N⁺(R′)₂—, —P(O)R′O—, —C(O)NR′—, —SO₂NR′—, —OP(O)R′O—, —P(O)(N(R′)₂)NR′—, —P(R′)₂═N— or —P(O)R′—, where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl and
X denotes F, Cl, Br or I;
phosphonium cations of the formula (3)

[P(R²)₄]⁺ (3),

where
R² in each case, independently of one another,
denotes H, OR′ or N(R′)₂,
denotes a linear or branched alkyl group having 1 to 20 C atoms,
denotes a linear or branched alkenyl group having 2 to 20 C atoms and at least one double bond,
denotes a linear or branched alkynyl group having 2 to 20 C atoms and at least one triple bond or
denotes a saturated, partially unsaturated, unsaturated or aromatic cyclic group having 3 to 7 C atoms, which may be mono- or polysubstituted by a linear or branched alkyl group having 1 to 6 C atoms,
where one or two R² may be partially replaced by halogen, preferably —F and/or —Cl, and/or by —OR′, —CN, —N(R′)₂, —C(O)OR′, —C(O)R′, —C(O)N(R′)₂, —SO₂N(R′)₂, —C(O)X, —SR′, —S(O)R′, —SO₂R′ and where one or two carbon atoms of the radical R² which are not adjacent and are not in the α-position may be replaced by atoms and/or atom groups selected from the group —O—, —S—, —S(O)—, —SO₂—, —SO₂O—, —C(O)—, —C(O)O—, —N⁺(R′)₂—, —P(O)R′O—, —C(O)NR′—, —SO₂NR′—, —OP(O)R′O—, —P(O)(N(R′)₂)NR′—, —P(R′)₂═N— or —P(O)R′—, where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl and
X denotes F, Cl, Br or I;
thiouronium cations of the formula (4),

[C(NR³R⁴)(SR⁵)(NR⁶R⁷)]⁺ (4),

where
R³ to R⁷ each, independently of one another,
denote H, where H is excluded for R⁵,
denotes a linear or branched alkyl group having 1 to 20 C atoms,
denotes a linear or branched alkenyl group having 2 to 20 C atoms and at least one double bond,
denotes a linear or branched alkynyl group having 2 to 20 C atoms and at least one triple bond or
denotes a saturated, partially unsaturated, unsaturated or aromatic cyclic group having 3 to 7 C atoms, which may be mono- or polysubstituted by a linear or branched alkyl group having 1 to 6 C atoms,
where one or two of the substituents R³ to R⁷ may be partially replaced by halogen, preferably —F and/or —Cl, and/or by —OR′, —CN, —N(R′)₂, —C(O)OR′, —C(O)R′, —C(O)N(R′)₂, —SO₂N(R′)₂, —C(O)X, —SR′, —S(O)R′, —SO₂R′ and where one or two carbon atoms of the radicals R³ to R⁷ which are not adjacent and are not in the α-position may be replaced by atoms and/or atom groups selected from the group —O—, —S—, —S(O)—, —SO₂—, —SO₂O—, —C(O)—, —C(O)O—, —N⁺(R′)₂—, —P(O)R′O—, —C(O)NR′—, —SO₂NR′—, —OP(O)R′O—, —P(O)(N(R′)₂)NR′—, —P(R′)₂═N— or —P(O)R′—, where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl and
X denotes F, Cl, Br or I;
guanidinium cations of the formula (5)

[C(NR⁸R⁹)(NR¹⁰R¹¹)(NR¹²R¹³)]⁺ (5),

where
R⁸ to R¹³ each, independently of one another,

### H, —CN, N(R′)₂, —OR′,

denotes a linear or branched alkyl group having 1 to 20 C atoms,
denotes a linear or branched alkenyl group having 2 to 20 C atoms and at least one double bond,
denotes a linear or branched alkynyl group having 2 to 20 C atoms and at least one triple bond or denotes a saturated, partially unsaturated, unsaturated or aromatic cyclic group having 3 to 7 C atoms, which may be mono- or polysubstituted by a linear or branched alkyl group having 1 to 6 C atoms,
where one or two of the substituents R⁸ to R¹³ may be partially replaced by halogen, preferably —F and/or —Cl, and/or by —OR′, —CN, —N(R′)₂, —C(O)OR′, —C(O)R′, —C(O)N(R′)₂, —SO₂N(R′)₂, —C(O)X, —SR′, —S(O)R′, —SO₂R′ and where one or two carbon atoms of the radicals R⁸ to R¹³ which are not adjacent and are not in the α-position may be replaced by atoms and/or atom groups selected from the group —O—, —S—, —S(O)—, —SO₂—, —SO₂O—, —C(O)—, —C(O)O—, —N⁺(R′)₂—, —P(O)R′—, —C(O)NR′—, —SO₂NR′—, —OP(O)R′O—, —P(O)(N(R′)₂)NR′—, —P(R′)₂═N— or —P(O)R′—, where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl and
X denotes F, Cl, Br or I;
heterocyclic cations of the formula (6),

[Het]^{z+} (6),

where
HetN^{z+} denotes a heterocyclic cation selected from the group

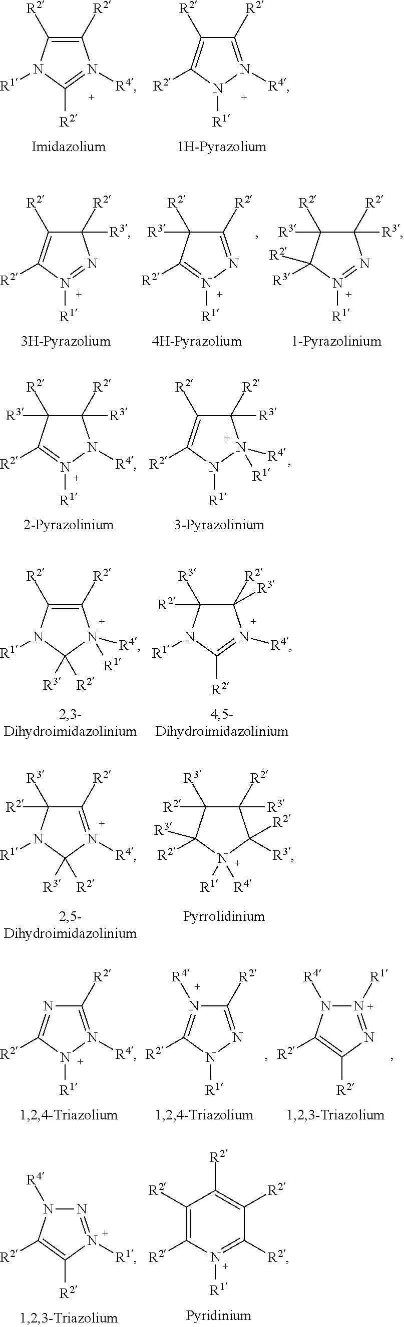

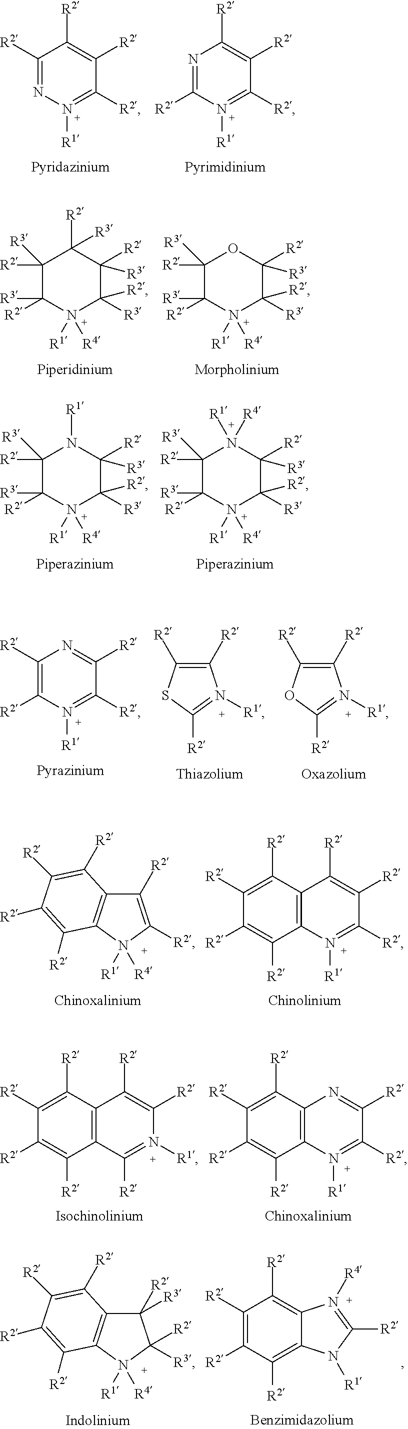

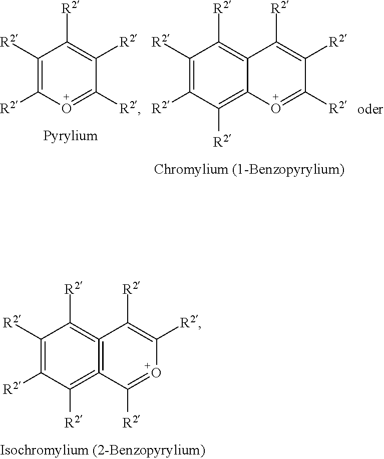

where the substituents
R^{1′} to R^{4′} each, independently of one another, denote

### H,

linear or branched alkyl group having 1 to 20 C atoms,
linear or branched alkenyl group having 2 to 20 C atoms and at least one double bond,
linear or branched alkynyl group having 2 to 20 C atoms and at least one triple bond,
cycloalkyl group having 3 to 7 C atoms, which may be mono- or polysubstituted by a linear or branched alkyl group having 1 to 6 C atoms,
unsubstituted phenyl or phenyl which is mono- or polysubstituted by a linear or branched alkyl group,
heteroaryl, heteroaryl-C₁-C₆-alkyl or aryl-C₁-C₆-alkyl and
R^{2′} may additionally denote F, Cl, Br, I, —CN, —OR′, —N(R′)₂, —P(O)(R′)₂, —P(O)(OR′)₂, —P(O)(N(R′)₂)₂, —C(O)R′, —C(O)OR′, —C(O)X, —C(O)N(R′)₂, —SO₂N(R′)₂, —SO₂OH, —SO₂X, —SR′, —S(O)R′ and/or —SO₂R′, with the assumption that the substituents R^{1′}, R^{3′}, R^{4′} then each, independently of one another, denote H and/or a linear or branched alkyl group having 1 to 20 C atoms and/or a linear or branched alkenyl group having 2 to 20 C atoms and at least one double bond,
where the substituents R^{1′}, R², R^{3′} and/or R^{4′} together may also form a ring system,
where one or more substituents R¹ to R⁴ may also be partially replaced by halogen, preferably F and/or Cl, and/or by —OR′, N(R′)₂, —CN, —C(O)OR′, —C(O)R′, —C(O)N(R′)₂, —SO₂N(R′)₂, —C(O)X, —SR′, —S(O)R′, —SO₂R′, but where R^{1′} and R^{4′} cannot simultaneously be fully substituted by halogen and where one or two carbon atoms of the radicals R^{1′} to R^{4′} which are not adjacent and are not in the α-position may be replaced by atoms and/or atom groups selected from the group —O—, —S—, —S(O)—, —SO₂—, —SO₂O—, —C(O)—, —C(O)O—, —N⁺(R′)₂—, —P(O)R′O—, —C(O)NR′—, —SO₂NR′—, —OP(O)R′O—, —P(O)(N(R′)₂)NR′—, —P(R′)₂═N— or —P(O)R′—, where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl and
X denotes F, Cl, Br or I or
iodonium cations of the formula (7)

Ar-I⁺-Ar (7),

where
Ar in each case, independently of one another, denotes an aryl group having 6 to 30 C atoms, which may be unsubstituted or substituted by at least one linear or branched alkenyl group having 2 to 20 C atoms and at least one double bond, a linear or branched alkynyl group having 2 to 20 C atoms and at least one triple bond and/or by R¹*, SR′^{′}, N(R′)₂, CN and/or halogen, where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl and
R¹* in each case, independently of one another, denotes unfluorinated or partially fluorinated linear or branched alkyl group having 1 to 8 C atoms, saturated cycloalkyl group having 3 to 7 C atoms or unsubstituted or substituted phenyl and x denotes F, Cl, Br or I.

A linear or branched alkyl group having 1 to 20 C atoms is, for example, methyl, ethyl, isopropyl, propyl, butyl, sec-butyl or tert-butyl, furthermore pentyl, 1-, 2- or 3-methylbutyl, 1,1-, 1,2- or 2,2-dimethylpropyl, 1-ethylpropyl, n-hexyl, n-heptyl, n-octyl, furthermore nonyl, decyl, undecyl, dodecyl, tridecyl, tetradecyl, pentadecyl, hexadecyl, heptadecyl, octadecyl, nonadecyl and eicosyl. From the linear or branched alkyl group having 1 to 20 C atoms, the linear or branched alkyl group having 1 to 8 C atoms, i.e. methyl, ethyl, isopropyl, propyl, butyl, sec-butyl or tert-butyl, furthermore pentyl, 1-, 2- or 3-methylbutyl, 1,1-, 1,2- or 2,2-dimethylpropyl, 1-ethylpropyl, n-hexyl, n-heptyl, n-octyl, is preferably selected. From the linear or branched alkyl group having 1 to 20 C atoms, the straight-chain or branched alkyl group having 1 to 4 C atoms, i.e. methyl, ethyl, isopropyl, propyl, butyl, sec-butyl or tert-butyl, is particularly preferably selected. If the alkyl group is partially fluorinated, at least one H atom is replaced by an F atom. If it is perfluorinated, all H atoms of the corresponding alkyl group have been replaced by F atoms.

A linear or branched alkenyl group having 2 to 20 C atoms, where a plurality of double bonds may also be present, is, for example, vinyl, allyl, 2- or 3-butenyl, isobutenyl, sec-butenyl, furthermore 4-pentenyl, isopentenyl, hexenyl, heptenyl, octenyl, —C₉H₁₇, —C₁₀H₁₉ to —C₂₀H₃₉; preferably allyl, 2- or 3-butenyl, isobutenyl, sec-butenyl, preference is furthermore given to 4-pentenyl, isopentenyl or hexenyl. If the alkenyl group is partially fluorinated, at least one H atom is replaced by an F atom. If it is perfluorinated, all H atoms of the corresponding alkenyl group have been replaced by F atoms.

A linear or branched alkynyl group having 2 to 20 C atoms, where a plurality of triple bonds may also be present, is, for example, ethynyl, 1- or 2-propynyl, 2- or 3-butynyl, furthermore 4-pentynyl, 3-pentynyl, hexynyl, heptynyl, octynyl, —C₉H₁₅, —C₁₀H₁₇ to —C₂₀H₃₇, preferably ethynyl, 1- or 2-propynyl, 2- or 3-butynyl, 4-pentynyl, 3-pentynyl or hexynyl. If the alkynyl group is partially fluorinated, at least one H atom is replaced by an F atom. If it is perfluorinated, all H atoms of the corresponding alkynyl group have been replaced by F atoms.

Halogen denotes F, Cl, Br or I, particularly preferably F or Cl.

An unsubstituted saturated or partially unsaturated, unsaturated or aromatic cyclic group having 3-7 C atoms is, for example, cyclopropyl, cyclobutyl, cyclopentyl, cyclohexyl, cycloheptyl, cyclopentenyl, cyclohexenyl, cycloheptenyl or phenyl, each of which may be mono- or polysubstituted by linear or branched C₁- to C₆-alkyl groups.

Aryl having 6 to 12 C atoms denotes an aryl group having 6 to 12 C atoms and is, for example, phenyl, naphthyl or anthracenyl, which may be unsubstituted or substituted by a straight-chain or branched alkyl group having 1 to 6 C atoms. The substitution may take place one or more times by the substituents indicated, preferably once. The phenyl group is preferably substituted in the 4-position. Aryl having 6 to 12 C atoms is preferably phenyl, which may be substituted by at least one straight-chain or branched alkyl group having 1 to 6 C atoms. In the case of the sulfonium cations of the formula (2), the phenyl group is preferably substituted by SR′.

Aryl having 6 to 30 C atoms denotes an aryl group having 6 to 30 C atoms and is an aromatic group having a common aromatic electron system having 6 to 30 C atoms, optionally mono- or polysubstituted by R¹*, OR′, N(R′)₂, CN or halogen. An aryl group having 6 to 34 C atoms, preferably having 6 to 24 C atoms, is preferably 1-, 2-, 3-, 4-, 5- or 6-phenyl, 1-, 2-, 3-, 4-, 6-, 7- or 8-naphthyl, 1-, 2-, 3-, 4-, 6-, 7- or 8-phenanthrenyl, 1-, 2-, 3-, 4-, 5-, 6-, 7-, 8-, 9- or 10-anthracenyl, 1-, 2-, 3-, 4-, 5-, 6-, 7-, 8-, 9-, 10-, 11- or 12-tetracenyl, 1-, 2-, 3-, 4-, 5-, 6-, 7-, 8-, 9-, 10-, 11- or 12-benz[a]anthracenyl, 1-, 2-, 3-, 4-, 5-, 6-, 7-, 8-, 9-, 10-, 11-, 12-, 13- or 15-pentacenyl, 1-, 2-, 3-, 4-, 5-, 6-, 7-, 8-, 9-, 10-, 11- or 12-chrysenyl, 1-, 2-, 3-, 4-, 5-, 6-, 7-, 8-, 9- or 10-pyrenyl, 1-, 2-, 3-, 4-, 5-, 6-, 7-, 8-, 9-, 10-, 11- or 12-benzo[a]pyrenyl, 1-, 2-, 3-, 4-, 5-, 6-, 7- or 8-azulenyl, 1-, 2-, 3-, 4-, 5-, 6-, 7-, 8-, 9- or 10-fluoranthenyl, 1-, 2-, 3-, 4-, 5-, 6-, 7-, 8-, 9-, 10-, 11- or 12-perylenyl, 1-, 2-, 3-, 4-, 5-, 6- or 7-indenyl or 1-, 2-, 3-, 4-, 5-, 6-, 7-, 8- or 9-fluorenyl, each of which is substituted by R¹*, OR′, N(R′)₂, CN or halogen or unsubstituted.

Aryl-C₁-C₆-alkyl denotes, for example, benzyl, phenylethyl, phenylpropyl, phenylbutyl, phenylpentyl or phenylhexyl, where both the phenyl ring and also the alkylene chain, as described above, may be partially or fully substituted by halogens, in particular —F and/or —Cl, or partially by —OR′, —N(R′)₂, —CN, —C(O)N(R′)₂, —SO₂N(R′)₂, where R′ has a meaning described above or described below.

Suitable substituents R and R² to R¹³ of the cations of the formulae (2) to (5) are, in accordance with the invention, preferably: H, straight-chain or branched C₁- to C₂₀—, in particular straight-chain or branched C₁- to C₁₄-alkyl groups, saturated C₃- to C₇-cycloalkyl groups, which may be substituted by straight-chain or branched C₁- to C₆-alkyl groups, or phenyl, which may be substituted by straight-chain or branched C₁- to C₆-alkyl groups.

The substituents R and R² in the cations of the formula (2) or (3) may be identical or different here. In the case of cations of the formula (2), preferably all substituents R are identical or three are identical and one substituent is different. In the case of cations of the formula (3), preferably three or four substituents R² are identical.

The substituents R and R² are especially preferably methyl, ethyl, 2-methoxyethyl, ethoxymethyl, 2-ethoxyethyl, isopropyl, 3-methoxypropyl, propyl, n-butyl, sec-butyl, n-pentyl, n-hexyl, n-octyl, n-decyl or n-tetradecyl. If the alkyl group is not designated further by n or iso, the n-alkyl group is used.

Preferred ammonium cations of the formula (2) are, for example, tetramethylammonium, tetraethylammonium, tetrapropylammonium, tetrabutylammonium, trimethyl(ethyl)ammonium, triethyl(methyl)ammonium, tripropyl(methyl)ammonium, tributyl(methyl)ammonium, tripentyl(methyl)ammonium, trihexyl(methyl)ammonium, triheptyl(methyl)ammonium, trioctyl(methyl)ammonium, trinonyl(methyl)ammonium, tridecyl(methyl)ammonium, trihexyl(ethyl)ammonium, ethyl(trioctyl)ammonium, propyl(dimethyl)ethylammonium, butyl(dimethyl)ethylammonium, methoxyethyl(dimethyl)ethylammonium, methoxyethyl(diethyl)methylammonium, methoxyethyl(dimethyl)propylammonium, ethoxyethyl(dimethyl)ethylammonium. A particularly preferred quaternary ammonium cation of the formula (3) is tributyl(methyl)ammonium.

Preferred tetraalkylphosphonium cations of the formula (3) are, for example, trimethyl(ethyl)phosphonium, triethyl(methyl)phosphonium, tripropyl(methyl)phosphonium, tributyl(methyl)phosphonium, tripentyl(methyl)phosphonium, trihexyl(methyl)phosphonium, triheptyl(methyl)phosphonium, trioctyl(methyl)phosphonium, trinonyl(methyl)phosphonium, tridecyl(methyl)phosphonium, trihexyl(ethyl)phosphonium, ethyl(trioctyl)phosphonium, propyl(dimethyl)ethylphosphonium, butyl(dimethyl)ethylphosphonium, methoxyethyl(dimethyl)ethylphosphonium, methoxyethyl(diethyl)methylphosphonium, methoxyethyl(dimethyl)propyl phosphonium, ethoxyethyl(dimethyl)ethyl phosphonium. Particularly preferred quaternary phosphonium cations are propyl(dimethyl)ethyl phosphonium and/or methoxyethyl(dimethyl)ethylphosphonium.

The substituents R⁰ in the cations of the formula (1) may likewise be identical or different.

In the case of cations of the formula (1), at least one substituent R⁰ is preferably phenyl or substituted phenyl. In the case of cations of the formula (1), the substituents R⁰ are preferably phenyl and/or SR′-substituted phenyl, where R′ has a meaning indicated above or a preferred meaning.

Preferred cations of the formula (1) are triphenylsulfonium, diphenyltolylsulfonium, diphenylethylsulfonium, diphenyl-2,2,2-trifluoroethyl sulfonium, diphenyl-2-ethoxyethylsulfonium, diphenyl-2-chloroethylsulfonium, diphenyl-3-bromopropylsulfonium, diphenyl-3-chloropropylsulfonium, diphenyl-3-cyanopropylsulfonium, diphenylallylsulfonium, diphenyl-4-pentenylsulfonium, diphenylpropargylsulfonium, diphenylbenzylsulfonium, diphenyl(p-cyanobenzyl)sulfonium, diphenyl(p-methylbenzyl)sulfonium, diphenyl(p-phenylthiobenzyl)sulfonium, diphenyl(3,3-dicyano-2-phenyl-2-propenyl)sulfonium, diphenyl(p-methylphenacyl)sulfonium, diphenyl(ethylcarboxy)methylsulfonium, diphenyl(n-octyl)sulfonium, diphenyl(n-octadecyl)sulfonium, diphenyl(ω-carboxytridecyl)sulfonium, diphenyl(3-oxypropyl)sulfonium, diphenyl(ω-carboxydodecyl)sulfonium, dihexylphenylsulfonium, ditolylphenylsulfonium, tritolylsulfonium, m- or p-(tert-butyl)phenyldiphenylsulfonium, m- or p-methoxyphenyldiphenylsulfonium, m- or p-CN-phenyldiphenylsulfonium, m- or p-C₆H₁₃S-phenyldiphenylsulfonium, m- or p-C₆H₅S-phenyldiphenylsulfonium, tri(p-methoxyphenyl)sulfonium, tri[4-(4-acetylphenylsulfanyl)phenyl]sulfonium, tri(4-tert-butylphenyl)sulfonium.

Up to four substituents of the thiouronium cation [C(NR³R⁴)(YR⁵)(NR⁶R⁷)]⁺ where Y═S may also be connected in pairs in such a way that mono-, bi- or polycyclic molecules form.

Without restricting generality, the formula (4) encompasses the following examples:

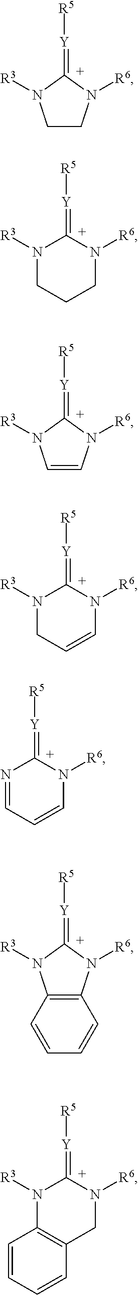

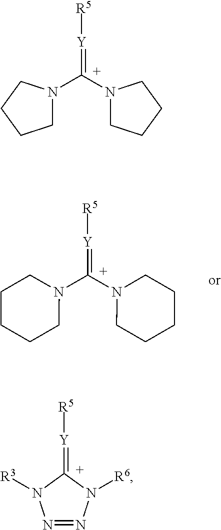

in which Y═S
and where the substituents R³, R⁵ and R⁶ may have a meaning indicated above or a particularly preferred meaning.

The carbocycles or heterocycles of the thiouronium cations indicated above may optionally be partially substituted by halogen, —OR′, —CN, —N(R′)₂, —C(O)OR′, —OC(O)R′, —OC(O)OR′, —C(O)R′, —C(O)N(R′)₂, —SO₂N(R′)₂, —C(O)X, —SR′, —S(O)R′ and/or —SO₂R′, where R′ and X have a meaning indicated above.

Up to four substituents of the guanidinium cation [C(NR⁸R⁹)(NR¹⁰R¹¹)—(NR¹²R¹³)]⁺ may also be connected in pairs in such a way that mono-, bi- or polycyclic molecules form.

Without restricting generality, the formula (7) encompasses the following examples:

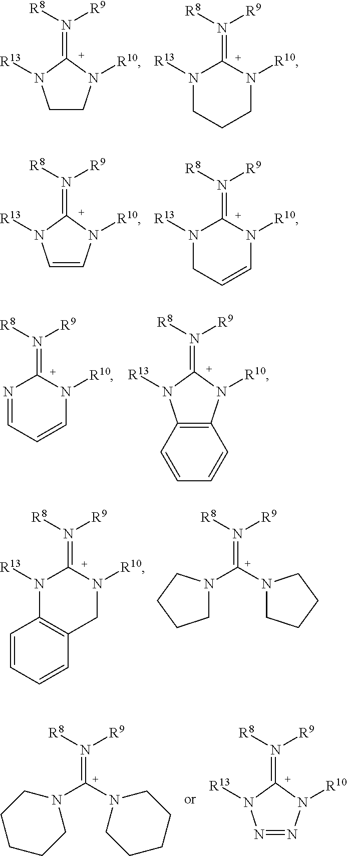

where the substituents R⁸ to R¹⁰ and R¹³ may have a meaning indicated above or a particularly preferred meaning.

The carbocycles or heterocycles of the guanidinium cations indicated above may optionally be fully substituted by halogen or partially substituted by halogen, —OR′, —CN, —N(R′)₂, —C(O)OR′, —OC(O)R′, —OC(O)OR′, —C(O)R′, —C(O)N(R′)₂, —SO₂N(R′)₂, —C(O)X, —SR′, —S(O)R′ and/or —SO₂R′, where R′ and X have a meaning indicated above.

The substituents R³ to R¹³ are each, independently of one another, preferably a straight-chain or branched alkyl group having 1 to 10 C atoms. The substituents R³ and R⁴, R⁶ and R⁷, R⁸ and R⁹, R¹⁰ and R¹¹ and R¹² and R¹³ in compounds of the formulae (4) and (5) may be identical or different here. R³ to R¹³ are particularly preferably each, independently of one another, methyl, ethyl, 2-methoxyethyl, ethoxymethyl, n-propyl, 3-methoxy-propyl, isopropyl, n-butyl, tert-butyl, sec-butyl, phenyl or cyclohexyl, very particularly preferably methyl, ethyl, n-propyl, isopropyl or n-butyl.

Suitable substituents R^{1′} and R^{4′} of compounds of the formula (6) are, in accordance with the invention, preferably: straight-chain or branched C₁- to C₂₀, in particular straight-chain or branched C₁- to C₁₂-alkyl groups, saturated C₃- to C₇-cycloalkyl groups, which may be substituted by straight-chain or branched C₁- to C₆-alkyl groups, or phenyl, which may be substituted by straight-chain or branched C₁- to C₆-alkyl groups.

Suitable substituents R^{2′} and R^{3′} of compounds of the formula (6) are, in accordance with the invention, besides H, preferably: straight-chain or branched C₁- to C₂₀, in particular straight-chain or branched C₁- to C₁₂-alkyl groups.

The substituents R^{1′} and R^{4′} are each, independently of one another, especially preferably methyl, ethyl, isopropyl, propyl, butyl, sec-butyl, pentyl, hexyl, octyl, decyl, cyclohexyl, phenyl or benzyl. They are very particularly preferably methyl, ethyl, n-butyl or n-hexyl. In pyrrolidinium, piperidinium or indolinium compounds, the two substituents R^{1′} and R^{4′} are preferably different.

The substituent R^{2′} or R^{3′} is in each case, independently of one another, in particular H, methyl, ethyl, isopropyl, propyl, butyl, sec-butyl, tert-butyl, cyclohexyl, phenyl or benzyl. R^{2′} is particularly preferably H, methyl, ethyl, isopropyl, n-propyl, n-butyl or sec-butyl. R^{2′} and R^{3′} are very particularly preferably H.

In R′, C₃- to C₇-cycloalkyl is, for example, cyclopropyl, cyclobutyl, cyclopentyl, cyclohexyl or cycloheptyl.

In R′, substituted phenyl denotes phenyl which is substituted by C₁- to C₆-alkyl, C₁- to C₆-alkenyl, F, Cl, Br, I, —C₁-C₆-alkoxy, —C(O)R″, —NR″₂, —SR″, —S(O)R″, —SO₂R″ or SO₂NR″₂, where R* denotes a non-, partially or perfluorinated C₁- to C₆-alkyl or C₃- to C₇-cycloalkyl, for example, o-, m- or p-methylphenyl, o-, m- or p-ethylphenyl, o-, m- or p-propylphenyl, o-, m- or p-isopropylphenyl, o-, m- or p-tert-butylphenyl, o-, m- or p-methoxyphenyl, o-, m- or p-ethoxyphenyl, o-, m- or p-fluorophenyl, o-, m- or p-chlorophenyl, o-, m- or p-bromophenyl, o-, m- or p-iodophenyl, further preferably 2,3-, 2,4-, 2,5-, 2,6-, 3,4- or 3,5-dimethylphenyl, 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,3-, 2,4-, 2,5-, 2,6-, 3,4- or 3,5-dimethoxyphenyl, 5-fluoro-2-methylphenyl, 3,4,5-trimethoxyphenyl or 2,4,5-trimethylphenyl.

In R^{1′} to R^{4′}, heteroaryl is taken to mean a saturated or unsaturated mono- or bicyclic heterocyclic radical having 5 to 13 ring members, where 1, 2 or 3 N and/or 1 or 2 S or O atoms may be present and the heterocyclic radical may be partially substituted by halogen, —OR¹, —CN, —N(R¹)₂, —C(O)OR¹, —OC(O)R¹, —OC(O)OR¹, —C(O)R¹, —C(O)N(R¹)₂, —SO₂N(R¹)₂, —C(O)X, —SR¹, —S(O)R¹ and/or —SO₂R¹, where R¹ and X have a meaning mentioned above.

The heterocyclic radical or Het is preferably substituted or unsubstituted 2- or 3-furyl, 2- or 3-thienyl, 1-, 2- or 3-pyrrolyl, 1-, 2-, 4- or 5-imidazolyl, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -4- or -5-yl, 1- or 5-tetrazolyl, 1,2,3-oxadiazol-4- or -5-yl 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 2-, 3-, 4-, 5- or 6-2H-thiopyranyl, 2-, 3- or 4-4H-thiopyranyl, 3- or 4-pyridazinyl, pyrazinyl, 2-, 3-, 4-, 5-, 6- or 7-benzofuryl, 2-, 3-, 4-, 5-, 6- or 7-benzothienyl, 1-, 2-, 3-, 4-, 5-, 6- or 7-1H-indolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 1-, 2-, 3-, 4-, 5-, 6-, 7- or 8-quinolinyl, 1-, 3-, 4-, 5-, 6-, 7- or 8-isoquinolinyl, 1-, 2-, 3-, 4- or 9-carbazolyl, 1-, 2-, 3-, 4-, 5-, 6-, 7-, 8- or 9-acridinyl, 3-, 4-, 5-, 6-, 7- or 8-cinnolinyl, 2-, 4-, 5-, 6-, 7- or 8-quinazolinyl or 1-, 2- or 3-pyrrolidinyl.

Heteroaryl-C₁-C₆-alkyl is now taken to mean, analogously to aryl-C₁-C₆-alkyl, for example, pyridinylmethyl, pyridinylethyl, pyridinylpropyl, pyridinylbutyl, pyridinylpentyl, pyridinylhexyl, where furthermore the heterocycles described above may be linked to the alkylene chain in this way.

HetN⁺ is preferably

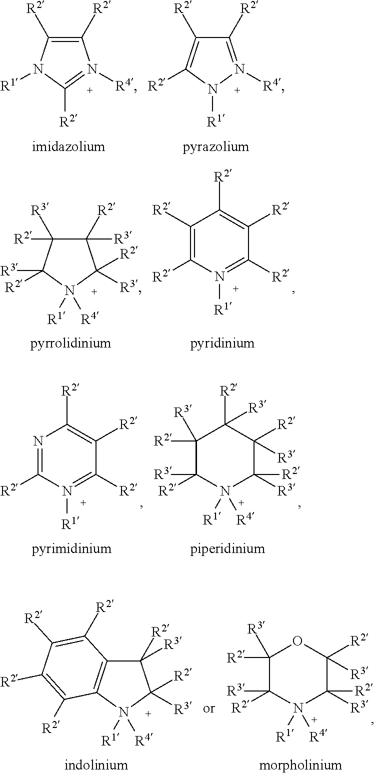

where the substituents R^{1′} to R^{4′} each, independently of one another, have a meaning described above.

Suitable for industrial applications of the compounds of the formula I as anticorrosion additive are compounds of the formula I whose cations conform to the formulae (2), (3), (4), (5) and (6), as described above or having substituents which are described as preferred. Especially suitable are compounds of the formula I whose cations conform to the formula (2) or to the formula (6) or cations of the formula (2) or of the formula (6) preferably indicated.

Preferred cations of the formula (6) are selected, for example, from 1,1-dialkylpyrrolidinium cations, 1-alkyl-1-alkoxyalkylpyrrolidinium cations, 1,3-dialkylimidazolium cations or 1-alkenyl-3-alkylimidazolium cations.

Preferred 1,1-dialkylpyrrolidinium cations are, for example, 1,1-dimethylpyrrolidinium, 1-methyl-1-ethylpyrrolidinium, 1-methyl-1-propylpyrrolidinium, 1-methyl-1-butylpyrrolidinium, 1-methyl-1-pentylpyrrolidinium, 1-methyl-1-hexylpyrrolidinium, 1-methyl-1-heptylpyrrolidinium, 1-methyl-1-octylpyrrolidinium, 1-methyl-1-nonylpyrrolidinium, 1-methyl-1-decylpyrrolidinium, 1,1-diethylpyrrolidinium, 1-ethyl-1-propylpyrrolidinium, 1-ethyl-1-butylpyrrolidinium, 1-ethyl-1-pentylpyrrolidinium, 1-ethyl-1-hexylpyrrolidinium, 1-ethyl-1-heptylpyrrolidinium, 1-ethyl-1-octylpyrrolidinium, 1-ethyl-1-nonylpyrrolidinium, 1-ethyl-1-decylpyrrolidinium, 1,1-dipropylpyrrolidinium, 1-propyl-1-methylpyrrolidinium, 1-propyl-1-butylpyrrolidinium, 1-propyl-1-pentylpyrrolidinium, 1-propyl-1-hexylpyrrolidinium, 1-propyl-1-heptylpyrrolidinium, 1-propyl-1-octylpyrrolidinium, 1-propyl-1-nonylpyrrolidinium, 1-propyl-1-decylpyrrolidinium, 1,1-dibutylpyrrolidinium, 1-butyl-1-methylpyrrolidinium, 1-butyl-1-pentylpyrrolidinium, 1-butyl-1-hexylpyrrolidinium, 1-butyl-1-heptylpyrrolidinium, 1-butyl-1-octylpyrrolidinium, 1-butyl-1-nonylpyrrolidinium, 1-butyl-1-decylpyrrolidinium, 1,1-dipentylpyrrolidinium, 1-pentyl-1-hexylpyrrolidinium, 1-pentyl-1-heptylpyrrolidinium, 1-pentyl-1-octylpyrrolidinium, 1-pentyl-1-nonylpyrrolidinium, 1-pentyl-1-decylpyrrolidinium, 1,1-dihexylpyrrolidinium, 1-hexyl-1-heptylpyrrolidinium, 1-hexyl-1-octylpyrrolidinium, 1-hexyl-1-nonylpyrrolidinium, 1-hexyl-1-decylpyrrolidinium, 1,1-dihexylpyrrolidinium, 1-hexyl-1-heptylpyrrolidinium, 1-hexyl-1-octylpyrrolidinium, 1-hexyl-1-nonylpyrrolidinium, 1-hexyl-1-decylpyrrolidinium, 1,1-diheptylpyrrolidinium, 1-heptyl-1-octylpyrrolidinium, 1-heptyl-1-nonylpyrrolidinium, 1-heptyl-1-decylpyrrolidinium, 1,1-dioctylpyrrolidinium, 1-octyl-1-nonylpyrrolidinium, 1-octyl-1-decylpyrrolidinium, 1-1-dinonylpyrrolidinium, 1-nonylecylpyrrolidinium or 1,1-didecylpyrrolidinium. Very particular preference is given to 1-butyl-1-methylpyrrolidinium or 1-propyl-1-methylpyrrolidinium.

Preferred 1-alkyl-1-alkoxyalkylpyrrolidinium cations are, for example, 1-methoxyethyl-1-methylpyrrolidinium, 1-methoxyethyl-1-ethylpyrrolidinium, 1-methoxyethyl-1-propylpyrrolidinium, 1-methoxyethyl-1-butylpyrrolidinium, 1-ethoxyethyl-1-methylpyrrolidinium, 1-ethoxymethyl-1-methylpyrrolidinium. Very particular preference is given to 1-methoxyethyl-1-methylpyrrolidinium.

Preferred 1,3-dialkylimidazolium cations are, for example, 1-ethyl-3-methylimidazolium, 1-methyl-3-propylimidazolium, 1-butyl-3-methylimidazolium, 1-methyl-3-pentylimidazolium, 1-ethyl-3-propylimidazolium, 1-butyl-3-ethylimidazolium, 1-ethyl-3-pentylimidazolium, 1-butyl-3-propylimidazolium, 1,3-dimethylimidazolium, 1,3-diethylimidazolium, 1,3-dipropypylimidazolium, 1,3-dibutylimidazolium, 1,3-dipentylimidazolium, 1,3-dihexylimidazolium, 1,3-diheptylimidazolium, 1,3-dioctylimidazolium, 1,3-dinonylimidazolium, 1,3-didecylimidazolium, 1-hexyl-3-methylimidazolium, 1-heptyl-3-methylimidazolium, 1-methyl-3-octylimidazolium, 1-methyl-3-nonylimidazolium, 1-decyl-3-methylimidazolium, 1-ethyl-3-hexylimidazolium, 1-ethyl-3-heptylimidazolium, 1-ethyl-3-octylimidazolium, 1-ethyl-3-nonylimidazolium or 1-decyl-3-ethylimidazolium. Particularly preferred cations are 1-ethyl-3-methylimidazolium, 1-butyl-3-methylimidazolium or 1-methyl-3-propylimidazolium.

Particularly preferred 1-alkenyl-3-alkylimidazolium cations are 1-allyl-3-methylimidazolium or 1-allyl-2,3-dimethylimidazolium.

Very particularly preferred compounds of the formula I as anticorrosion additives are compounds whose cations conform to the formula (3) or correspond to the preferred or particularly preferred cations of the formula (3).

Preferred cations of the formula (7) are diphenyliodonium, ditolyliodonium, phenyltolyliodonium, tolyl-(4-sec-butylphenyl)iodonium, di(p-tert-butylphenyl)iodonium, p-methoxyphenylphenyliodonium, di(p-methoxyphenyliodonium, m- or p-CN-phenylphenyliodonium, m- or p-(C₆H₅S)-phenylphenyliodonium.

For industrial use of a cationic polymerisation initiator, photopolymerisation initiator or photo-acid generator, preference is given to compounds of the formula I whose cations conform to the formulae (1), (4) or (7) or correspond to the cations pyrylium, 1-benzopyrylium or 2-benzopyrylium, as described above or having substituents which are described as preferred or having the disclosed cations indicated as preferred.

Particularly preferred organic cations of the formula I for industrial applications as cationic polymerisation initiator, photopolymerisation initiator are accordingly triarylsulfonium or diaryliodonium ions.

Very particularly preferred organic cations for industrial applications as cationic polymerisation initiator, photopolymerisation initiator or photo-acid generator are triphenylsulfonium, tritolylsulfonium, p-(tert-butyl)phenyldiphenylsulfonium, p-methoxyphenyldiphenylsulfonium, p-C₆H₁₃S-phenyldiphenylsulfonium, m- or (p-C₆H₅S-phenyl)diphenylsulfonium, tri[4-(4-acetylphenylsulfanyl)phenyl]sulfonium, tri(4-tert-butylphenyl)sulfonium, diphenyliodonium, ditolyliodonium, phenyltolyliodonium, di(p-tert-butylphenyl)iodonium, m- or (p-C₆H₅S-phenyl)phenyliodonium, tolyl-(4-sec-butylphenyl)iodonium.

A suitable starting material for the synthesis of the compounds of the formula I are alcohols of the formula II,

H—(CF₂—CF₂)_{y}—CH₂—OH II,

where y denotes 1, 2 or 3.

Alcohols of the formula II of this type are commercially available or can be prepared by processes which are known to the person skilled in the art, for example based on Donald R. Baer, Industrial and Engineering Chemistry, Vol. 51, No. 7, 1959, 829-830.

The invention accordingly furthermore relates to a process for the preparation of compounds of the formula I, as described above or described as preferred and where x in formula I denotes 4, characterised in that an alcohol of the formula II

H—(CF₂—CF₂)_{y}—CH₂—OH II,

in which y denotes 1, 2 or 3,
in a suitable solvent is treated with sodium hydroxide solution and subsequently with 1,4-butanesultone, and the sodium salt obtained is optionally subjected to salt exchange in a metathesis reaction with compounds of the formula III,

KtA III,

to give the corresponding compound of the formula I,
where Kt denotes an inorganic or organic cation, where Na⁺ is excluded, and A is selected from the group of the anions Cl⁻, Br⁻, I⁻, [HF₂]⁻, [R₁SO₃]⁻, [R₂COO]⁻, [R₂SO₃]⁻, [R₁OSO₃]⁻, [PF₆]⁻, [BF₄]⁻, [SO₄]²⁻, [HSO₄]⁻, [NO₃]⁻, [(R₂)₂P(O)O]⁻, [R₂P(O)O₂]²⁻, [(R₁O)₂P(O)O]⁻, [(R₁O)P(O)O₂]²⁻, [(R₁O)R₁P(O)O]⁻, [HOCO₂]⁻ or [CO₃]²⁻, with the assumption that the anions [SO₄]²⁻ and [CO₃]²⁻ are used for the preparation of compounds of the formula I containing other alkali-metal cations and
where
R₁ in each case, independently of one another, denotes a linear or branched alkyl group having 1 to 12 C atoms and
R₂ in each case, independently of one another, denotes a linear or branched perfluorinated alkyl group having 1 to 12 C atoms and
where the electroneutrality of the salt KtA is observed.

1,4-Butanesultone is likewise commercially available. Suitable solvents can be selected from methanol, ethanol, 2-propanol or acetonitrile. A particularly suitable solvent is 2-propanol. A 50% NaOH solution is preferably used.

The reaction of the compound of the formula II with 1,4-butanesultone is preferably carried out at temperatures between 30° C. and 100° C., particularly preferably between 50° C. and 90° C., very particularly preferably at 80° C. The sodium salt formed from the reaction can also be purified before the metathesis reaction using methods which are known to the person skilled in the art, for example by recrystallisation. However, it is also possible to employ the sodium salt in the metathesis reaction without purification. The conditions of the metathesis reaction are described below.

The invention accordingly furthermore also relates to a process for the preparation of compounds of the formula I as described above or described as preferred and where x in formula I denotes 3, characterised in that an alcohol of the formula II

H—(CF₂—CF₂)_{y}—CH₂—OH II,

in which y denotes 1, 2 or 3,
is reacted with allyl chloride and sodium hydroxide solution in a suitable solvent and in the presence of a phase-transfer catalyst, the allyl ether of the formula IV,

H—(CF₂—CF₂)_{y}—CH₂—O—CH₂—CH═CH₂ IV,

in which y denotes 1, 2 or 3,
formed as an intermediate is reacted with an aqueous NaHSO₃/Na₂SO₃ solution at a pH of 7 to 8 in the presence of a solubiliser, and the sodium salt formed is subjected to salt exchange in a metathesis reaction with compounds of the formula III,

KtA III,

to give the corresponding compound of the formula I,
where Kt denotes an inorganic or organic cation, where Na⁺ is excluded, and A is selected from the group of the anions, Cl⁻, Br⁻, I⁻, [HF₂]⁻, [R₁SO₃]⁻, [R₂COO]⁻, [R₂SO₃]⁻, [R₁OSO₃]⁻, [PF₆]⁻, [BF₄]⁻, [SO₄]²⁻, [HSO₄]⁻, [NO₃]⁻, [(R₂)₂P(O)O]⁻, [R₂P(O)O₂]²⁻, [(R₁O)₂P(O)O]⁻, [(R₁O)P(O)O₂]²⁻, [(R₁O)R₁P(O)O]⁻, [HOCO₂]⁻ or [CO₃]²⁻, with the assumption that the anions [SO₄]²⁻ and [CO₃]²⁻ are used for the preparation of compounds of the formula I containing other alkali-metal cations and
where
R₁ in each case, independently of one another, denotes a linear or branched alkyl group having 1 to 12 C atoms and
R₂ in each case, independently of one another, denotes a linear or branched perfluorinated alkyl group having 1 to 12 C atoms and
where the electroneutrality of the salt KtA is observed.

The conditions of the reaction to give compounds of the formula IV are known from Georg Sonnek et al, Journal of Organometallic Chemistry 1991, 405, 179-182:

The reaction to give the compounds of the formula IV is generally carried out without a further solvent. In general, a large excess of allyl chloride is used.

Suitable phase-transfer catalysts are, for example, tetraalkylammonium chloride, tetraalkylammonium hydrogensulfate or 3-siloxanylpropyl-ammonium halides, in particular tetrabutylammonium chloride, tetrabutylammonium hydrogensulfate or the compounds [(Me₃SiO)₃Si-pr-NMe₂-Pr]Br or [(Me₃SiO)₂MeSi-pr-NMe₂-Pr]Br, where Me denotes methyl, pr denotes propylene and Pr denotes propyl.

The reaction can advantageously be carried out under ultrasound.

The reaction temperature is between 20° C. to 60° C., advantageously at 40° C.

The further reaction of the compounds of the formula IV to give the corresponding sodium salts of the formula I is carried out with an aqueous NaHSO₃/Na₂SO₃ solution at a pH of 7 to 8 in the presence of a solubiliser, preferably at room temperature.

Suitable solubilisers are, for example, alcohols, such as methanol, ethanol, 2-propanol or n-butanol, or cyclic ethers, such as dioxane, preferably ethanol.

The reaction times can be shortened by addition of peroxides, for example (NH₄)₂S₂O₈, or by passing in air.

The subsequent salt-exchange reaction or also metathesis reaction, is carried out in water or advantageously in organic solvents, where the reaction in water are carried out at temperatures of 0°-50° C., preferably at 0°-25° C. The reaction is particularly preferably carried out at room temperature.

The reaction in organic solvents is carried out at temperatures between between −80° and 100° C. Suitable organic solvents are selected, for example, from acetonitrile, propionitrile, acetone, dioxane, dichloromethane, dimethoxyethane, dimethyl sulfoxide, tetrahydrofuran, dimethylformamide or dialkyl ethers, for example diethyl ether or methyl t-butyl ether, or alcohol, for example methanol, ethanol or isopropanol. The reaction is preferably carried out in acetonitrile, propionitrile, acetone, diethyl ether, dichlomethane or a mixture of acetonitrile/dichlomethane or acetonitrile/diethyl ether.

R₂ is in each case, independently of one another, preferably trifluoromethyl, pentafluoroethyl or nonafluorobutyl, particularly preferably trifluoromethyl or pentafluoroethyl.

R₁ is in each case, independently of one another, preferably methyl, ethyl or n-butyl, particularly preferably methyl or ethyl.

The compounds of the formula III, as described above are commercially available or can be prepared by processes known from the literature, for example as described in P. Wasserscheid, T. Welton (Eds.), Ionic Liquids in Synthesis, Second Edition, WILEY-VCH, Weinheim, 2008.

The anion of the compounds of the formula III is preferably Cl⁻, Br⁻, I⁻, [CH₃SO₃]⁻, [CF₃C(O)O]⁻, [CF₃SO₃]⁻, [CH₃OSO₃]⁻, [BF₄]⁻, [PF₆]⁻, [C₂H₅OSO₃]⁻, [(C₂F₅)₂P(O)O]⁻, [C₂F₅P(O)O₂]²⁻ or [SO₄]²⁻, particularly preferably Cl⁻, Br⁻, I⁻, [CH₃SO₃]⁻, [CH₃OSO₃]⁻, [CF₃COO]⁻, [CF₃SO₃]⁻, [(C₂F₅)₂P(O)O]⁻, [PF₆]⁻ or [BF₄]⁻, very particularly preferably Cl⁻.

The compounds of the formula I, as described above or described as preferred, which have the properties of an ionic liquid are also suitable as solvent or solvent additive, as catalyst or phase-transfer catalyst, as electrolyte constituent, as fluorosurfactant, as heat-exchange medium, as release agent or extractant, as plasticiser, as hydraulic fluid or additive for hydraulic fluids, as flameproofing agent or as additive in fire-extinguishing agents.

On use of the compounds of the formula I as solvent, these are suitable for any type of reaction known to the person skilled in the art, for example for transition metal- or enzyme-catalysed reactions, such as, for example, hydroformylation reactions, oligomerisation reactions, esterifications or isomerisations, where this list lays no claim to completeness.

On use as extractant, the compounds of the formula I can be used in order to separate off reaction products, but also in order to separate off impurities, depending on the solubility of the respective component in the ionic liquid. In addition, the ionic liquids can also serve as separation media in the separation of a plurality of components, for example in the distillative separation of a plurality of components of a mixture.

Further possible applications are the use as plasticiser in polymer materials, as flame retardant for a number of materials or applications and as additive in solar cells or in fuel cells.

Further areas of application of the compounds of the formula I which represent ionic liquids are solvents for carbohydrate-containing solids, in particular biopolymers and derivatives or degradation products thereof. A further area of application is the area of particle or nanomaterial synthesis, in which these ionic liquids are able to function as medium or additive.

The compounds of the formula I, as described above or described as preferred, are particularly suitable as anticorrosion additive, preferably in combination with a further ionic compound which does not conform to the formula I and is not a phosphate or alkylsulfate.

The invention therefore furthermore relates to the use of compounds of the formula I, as described above or described as preferred, as anticorrosion additives.

The invention therefore furthermore relates to the use of compounds of the formula I, as described above or described as preferred, together with ionic compounds which do not conform to the formula I and are not phosphates or alkylsulfates, in mechanical components, in chemical processes, in electronic components or everywhere where they come into contact with metals or metal alloys.

The compounds of the formula I, as described above or described as preferred, prevent or reduce, in particular, the corrosion of metals or metal alloys, in particular of nonferrous metals or of steels, particularly preferably of nonferrous metals.

The term metals or metal alloys encompasses both the group of nonferrous metals and also the group of light metals or the group of steels.

The term nonferrous metals is, in accordance with the invention, the collective term for a sub-group of non-iron metals excluding the noble metals, for example cadmium, cobalt, copper, nickel, lead, tin and zinc, as well as the alloys, such as brass, bronze and gunmetal.

The term light metals is generally applied to metals and alloys whose density is below 5 g/cm³, in particular this is aluminium in the sense of the invention.

The group of steels encompasses both construction steels and also stainless steels.

Stainless steel (in accordance with EN 10020) is a term for alloyed or unalloyed steels having a particular degree of purity, for example steels whose sulfur and phosphorus content does not exceed 0.025%.

The steel group numbers for stainless steels in accordance with EN 10027-2 know the terms 10-19 for unalloyed stainless steels and 20 to 89 for alloyed stainless steels.

By far the most frequent alloy components are chromium for chrome steel, chromium and nickel for chrome-nickel steel, molybdenum for molybdenum steel, together with chromium as chrome-molybdenum steel or also together with nickel, titanium with chrome and nickel for titanium steel and niobium for niobium steel.

In the characterisation of special steel with the material No. 1.4301, the number 43 according to the standard stands for “non-rusting”, with >2.5% of nickel, no molybdenum, niobium and titanium.

The group of steels therefore encompasses, for example, the stainless steels, characterised in accordance with DIN EN 10027-2, with the material numbers 1.4003 (X2CrNi12), 1.4006 (X12Cr13), 1.4016 (X6Cr17), 1.4021 (X20Cr13), 1.4104 (X14CrMoS17), 1.4301 (X5CrNi18-10), 1.4305 (X8CrNiS18-9), 1.4306 (X2CrNi19-11), 1.4307 (X2CrNi18-9), 1.4310 (X10CrNi18-8), 1.4316 (X1CrNi19-9), 1.4401 (X5CrNiMo17-12-2), 1.4404 (X2CrNiMo17-12-2), 1.4440 (X2CrNiMo19-12), 1.4435 (X2CrNiMo18-14-3) 1.4452 (X13CrMnMoN18-14-3), 1.4462 (X2CrNiMoN22-5-3), 1.4541 (X6CrNiTi18-10), 1.4571 (X6CrNiMoTi17-12-2), 1.4581 (GX5CrNiMoNb19-11-2), 1.4841 (X15CrNiSi25-21) and 1.7218 (25CrMo4). From this group, particular preference is given to the stainless steel with the material number 1.4301 (X5CrNi18-10).

Construction steels are low-carbon steels in which the carbon content is between 0% and 0.6%. The most-used types belong to the category of basic steels, are usually low-alloy and in some cases are heat-treated.

The group of steels therefore encompasses, for example, the basic steels characterised in accordance with DIN EN 10027-1, with the material numbers 5185 (previously St 33), S235JR (previously St 37-2), S235JRG1 (previously USt 37-2), S235JRG2 (previously RST 37-2), S275JR (previously St 44-2), S355JR, E295 (previously St 50-2), E335 (previously St 60-2), E360 (previously St 70-2).

The group of steels therefore encompasses, for example, the quality steels characterised in accordance with DIN EN 10027-1, with the material numbers S235JO (previously 1.0114), S235J2G3 (previously 1.0116) S235J2G4 (previously 1.0117), 275JO (previously 1.0143), S275J2G3 (previously 1.0144), S275J2G4 (previously 1.0145), S355JO (previously 1.0223), S355J2G3 (previously 1.0570) or S355J2G4 (previously 1.0577).

The group of steels therefore also encompasses, for example, roller-bearing steel with the designation 1.3505 100Cr6.

The compounds of the formula I are particularly suitable anticorrosion additives for the steels 100Cr6, St 37-2 and 1.4301 and copper and bronze.

The invention furthermore also relates to corresponding compositions comprising at least one compound of the formula I, as described above or described as preferred.

The compositions may include or comprise, essentially consist of or consist of the said requisite or optional constituents. All compounds or components which can be used in the compositions are either known and commercially available or can be synthesised by known processes.

Preferred compositions comprising compounds of the formula I, as described above or described as preferred, are lubricants or lubricating oils. The term lubricant composition or base oil also applies synonymously to lubricant compositions or lubricating grease compositions.

Particularly preferred compositions comprise at least one compound of the formula I, as described above or described as preferred, and at least one ionic compound which does not conform to the formula I and is not a phosphate or alkylsulfate.

The invention furthermore also relates to a process for the preparation of a composition comprising at least one compound of the formula I and at least one ionic compound, as described above, characterised in that the at least one compound of the formula I, as described above, described as preferred or the individual compounds, is mixed with the ionic compound which does not conform to the formula I and is not a phosphate or alkylsulfate and optionally with further additives for the desired application.

Preferred ionic compounds which does not conform to the formula I are ionic liquids which do not contain phosphate or alkylsulfate as anion.

The choice of the suitable ionic liquid, i.e. the correct choice of cation and anion, enables the respectively desired property of the composition, in particular of the lubricant or of the lubricating oil, to be established, such as, for example, the increase in the service life and lubricating action of the lubricant, the adjustment of the viscosity for improving the temperature suitability, the adjustment of the electrical conductivity for broadening the area of application. Suitable organic cations are the cations which have already been described above as cations of the formulae (1) to (7), and the particularly preferred cations of these formulae (1) to (7).

Particularly preferred cations for the ionic liquid are selected from the cations of the formulae (2), (3), (5) and (6) and the correspondingly preferably described cations of the formulae (2), (3), (5) and (6).

Preferred anions for the ionic liquid are selected from bis(perfluoroalkylsulfonyl)imide, perfluoroalkylsulfonate, tris(perfluoroalkyl)methide, bis(perfluoroalkyl)imide, bis(perfluoroaryl)imide, perfluoroarylperfluoroalkylsulfonylimide or tetracyanoborate. The perfluoroalkyl group of the anions is selected, for example, from trifluoromethyl, pentafluoroethyl, heptafluoropropyl or nonafluorobutyl. The perfluoroaryl group is, for example, pentafluorophenyl.

Particularly preferred anions for the ionic liquid are selected from trifluoromethanesulfonate, nonafluorobutylsulfonate or bis(trifluoromethanesulfonyl)imide.

The compounds of the formula I, as described above or described as preferred, are preferably combined with the following ionic liquids:
butylmethylpyrrolidinium bis(trifluoromethanesulfonyl)imide (BMPL NTF),
methylpropylpyrrolidinium bis(trifluoromethanesulfonyl)imide (MPPL NTF),
hexylmethylpyrrolidinium bis(trifluoromethanesulfonyl)imide (HMPL NTF),
1-ethyl-3-methylimidazolium bis(trifluoromethanesulfonyl)imide (EMIM NTF),
1-butyl-3-methylimidazolium bis(trifluoromethanesulfonyl)imide (BMIM NTF),
1-hexyl-3-methylimidazolium bis(trifluoromethanesulfonyl)imide (HMIM NTF),
1-butyl-2,3-dimethylimidazolium bis(trifluoromethanesulfonyl)imide (BMMIM NTF),
1-methyl-2,3-dimethylimidazolium bis(trifluoromethanesulfonyl)imide (MMMIM NTF),
1-ethyl-2,3-dimethylimidazolium bis(trifluoromethanesulfonyl)imide (EMMIM NTF),
1-propyl-2,3-dimethylimidazolium bis(trifluoromethanesulfonyl)imide (PMMIM NTF),
N-ethylpyridinium bis(trifluoromethanesulfonyl)imide (EPYR NTF),
N-hexylpyridinium bis(trifluoromethanesulfonyl)imide (HPYR NTF),
N-ethyl-3-methylpyridinium bis(trifluoromethanesulfonyl)imide (E3MPYR NTF),
trihexyltetradecylphosphonium bis(trifluoromethanesulfonyl)imide (PH3T NTF),
N-methyl-N,N,N-trioctylammonium bis(trifluoromethanesulfonyl)imide (NMO3 NTF),
N,N-diethyl-N-methyl-N-(2-methoxyethyl)bis(trifluoromethanesulfonyl)imide (NMOEEM NTF),
N-ethyl-3-methylpyridinium nonafluorobutanesulfonate (E3MPYR NON),
methylpropylpyrrolidinium nonafluorobutanesulfonate (MPPL NON),
hexylmethylpyrrolidinium nonafluorobutanesulfonate (HMPL NON),
1-ethyl-3-methylimidazolium nonafluorobutanesulfonate (EMIM NON),
1-butyl-3-methylimidazolium nonafluorobutanesulfonate (BMIM NON),
1-hexyl-3-methylimidazolium nonafluorobutanesulfonate (HMIM NON),
1-butyl-2,3-dimethylimidazolium nonafluorobutanesulfonate (BMMIM NON),
1-methyl-2,3-dimethylimidazolium nonafluorobutanesulfonate (MMMIM NON),
1-ethyl-2,3-dimethylimidazolium nonafluorobutanesulfonate (EMMIM NON),
1-propyl-2,3-dimethylimidazolium nonafluorobutanesulfonate (PMMIM NON),
N-ethylpyridinium nonafluorobutanesulfonate (EPYR NON),
N-hexylpyridinium nonafluorobutanesulfonate (HPYR NON),
butylmethylpyrrolidinium trifluoromethanesulfonate (BMPL OTF)
methylpropylpyrrolidinium trifluoromethanesulfonate (MPPL OTF),
hexylmethylpyrrolidinium trifluoromethanesulfonate (HMPL OTF),
1-ethyl-3-methylimidazolium trifluoromethanesulfonate (EMIM OTF),
1-butyl-3-methylimidazolium trifluoromethanesulfonate (BMIM OTF),
1-hexyl-3-methylimidazolium trifluoromethanesulfonate (HMIM OTF),
1-butyl-2,3-dimethylimidazolium trifluoromethanesulfonate (BMMIM OTF),
1-methyl-2,3-dimethylimidazolium trifluoromethanesulfonate (MMMIM OTF),
1-ethyl-2,3-dimethylimidazolium trifluoromethanesulfonate (EMMIM OTF),
1-propyl-2,3-dimethylimidazolium trifluoromethanesulfonate (PMMIM OTF),
N-ethylpyridinium trifluoromethanesulfonate (EPYR OTF),
N-hexylpyridinium trifluoromethanesulfonate (HPYR OTF),
N-ethyl-3-methylpyridinium trifluoromethanesulfonate (E3MPYR OTF).

Very particularly preferred ionic liquids in the compositions according to the invention or in the case of the use according to the invention are BMPL OTF, BMPL NTF, E3MPYR NON and BMMIM NTF.

The compositions according to the invention may also comprise further constituents.

A lubricant composition according to the invention may therefore be a base oil mixture, which can be adapted further to the application or may already be the usable lubricant composition or lubricating grease composition. The type and designation of the lubricant composition is then essentially determined by additives which are added to the lubricant composition.

Mention may be made by way of example of additives against corrosion, oxidation and for protection of metal influences, which as chelate compounds, free-radical scavengers, UV stabilisers, reaction layer formers, and inorganic or organic solid lubricants, such as polyimides, polytetrafluoroethylene, graphite, metal oxides, boron nitride, molybdenum sulfide and phosphate. In particular, additives are employed in the form of phosphorus- and sulfur-containing compounds, for example zinc dialkylthiophosphate, boric acid esters as antiwear/extreme pressure, aromatic amines, phenols, sulfur compounds as antioxidants, metal salts, esters, nitrogen-containing compounds, heterocyclic compounds as agents for corrosion prevention. Glycerol mono- or diesters can be employed as antifriction agents, and polyisobutylene, polyacrylate as viscosity improvers. The additives can be of an ionic structure or neutral.

Besides the anticorrosion additives of the formula I, as described above or preferably described, further corrosion inhibitors may also be present.

Examples of corrosion inhibitors are tolyltriazole (1H-4,5-methylbenzotriazole), alkanolamine salt (borates/carboxylates), ethoxyalkyl ether phosphate (for example polyethoxyalkyl(C8-C10) ether phosphate), ethynylcarbinol alkoxylate (for example propagyl alcohol alkoxylate(C2-C10)), mercaptothiadiazole (2,5-dimercapto-1,3,4-thiadiazole) or organic acids (for example ascorbic acid, oxalic acid, aspartic acid).

The compositions according to the invention can be mixed, for example, with other lubricants or with oils which have usual viscosities for industrial lubricants.

Suitable oils are synthetic oil, mineral oil and/or a native oil.

The synthetic oils are selected, for example, from an ester of an aliphatic or aromatic di-, tri- or tetracarboxylic acid with a C₇- to C₂₂-alcohol or C₇- to C₂₂-alcohols in the form of a mixture, from a polyphenyl ether or alkylated diphenyl ether, from an ester of trimethylolpropane, pentaerythritol or dipentaerythritol with aliphatic C₇- to C₂₂-carboxylic acids, from C₁₈-dimer acid esters with C₇- to C₂₂-alcohols, from complex esters, as individual components or in any desired mixture. Furthermore, the synthetic oil can be selected from poly-α-olefins, alkylated naphthalenes, alkylated benzenes, polyglycols, silicone oils or perfluoropolyethers.

The mineral oils can be selected from paraffin-basic, napthene-basic, aromatic hydrocracking oils or GTL liquids (GTL=gas to liquid, i.e. a process for the production of fuels from natural gas).

Native oils which can be used are triglycerides from animal/vegetable source, which can be refined by known processes, for example hydrogenation. The particularly preferred triglyceride oils are genetically modified triglyceride oils having a high oleic acid content. Typical genetically modified vegetable oils having a high oleic acid content which are used herein are safflower oil, corn oil, rapeseed oil, sunflower oil, soybean oil, linseed oil, peanut oil, lesquerella oil, meadowfoam oil and palm oil.

Possible additives are also thickeners, in particular for lubricating grease compositions. Examples of thickeners are products of the reaction of a diisocyanate with an amine or a diamine, as described in WO 2008/154997, metal soaps, metal sulfonates, metal complex soaps, bentonite, silicate powders, polytetrafluoroethylene (PTFE), polyamide or polyimide.

In general, compositions according to the invention, in particular lubricant compositions, comprise the at least one compound of the formula I in 0.05 to 5 per cent by weight, based on the entire composition. The compound of the formula I is particularly preferably present in 0.5 to 2 per cent by weight, very particularly preferably in 1 per cent by weight.

Preferred compositions comprise 5 to 95 per cent by weight of base oil mixture, 1 to 70 per cent by weight of at least one ionic compound, 3 to 50 per cent by weight of thickener and 0.1 to 10 per cent by weight of additives, the additives comprising at least one compound of the formula I as described above or described as preferred.

The compounds of the formula I, where the cations conform to the formula (1), (4) or (7) or the formulae for pyrylium, 1-benzopyrylium or 2-benzopyrylium, as described above or described as preferred, are particularly suitable as cationic polymerisation initiators, photopolymerisation initiators (or in other words as photoinitiator for polymerisations) or as photo-acid generators (PAG).

A cationic polymerisation initiator is capable of initiating a polymerisation of at least one monomer, for example the polymerisation of cationically polymerisable compounds, such as, for example, isobutylene, styrene, vinyl ethers, lactones, lactams, cyclic ethers or epoxy compounds.

In the case of a photopolymerisation initiator, the polymerisation process is initiated by irradiation of the initiator/monomer mixture, where energy beams of light, electrons or γ rays can be used for this purpose. Photopolymerisation generally results in a rapidly crosslinked end product. The compounds of the formula I containing the cations of the formula (1), (4) or (7) or the formulae for pyrylium, 1-benzopyrylium or 2-benzopyrylium, in particular the cations of the formula (1) and (7), are so-called cationic photoinitiators.

Photopolymerisation initiators are constituents of radiation-curing coating and resin formulations, which can often be cured in fractions of seconds by irradiation by light, laser, electrons or γ rays, in particular by UV light.

Photopolymerisation is widely used in various areas, for example for the curing of coating films, for the formation of planographic printing plates, synthetic resin relief printing plates and circuit boards, for the preparation of photoresists and photomasks in semiconductor technology, in particular using photolithography, for the production of black/white or coloured transfer films and dyeing foils.

If the compounds of the formula I, where the cations conform to the formula (1), (4) or (7) or the formulae for pyrylium, 1-benzopyrylium or 2-benzopyrylium, as described above or described as preferred, are irradiated with light, laser, electron beams or 7 rays, they are capable of generating the corresponding Brønsted acid or Lewis acid pointwise or in other words in catalytic amount and are able to initiate the polymerisation by the acid formed. However, the acid generated in catalytic amount may also effect deprotection of acid-sensitive organic functional groups in a compound. Compounds of this type are called photo-acid generators or abbreviated by PAG. Photo-acid generators are popular for use in compositions for the formation of photoresists and photosensitive materials.

The invention furthermore relates to a curable composition comprising at least one compound of the formula I, where the cations conform to the formula (1), (4) or (7) or the formulae for pyrylium, 1-benzopyrylium or 2-benzopyrylium, as described above or described as preferred, and at least one polymerisable compound.

The invention furthermore relates to a curable composition comprising at least one compound of the formula I, where the cations conform to the formula (1), (4) or (7) or the formulae for pyrylium, 1-benzopyrylium or 2-benzopyrylium, as described above or described as preferred, and at least one polymerisable compound.

Even without further comments, it is assumed that a person skilled in the art will be able to utilise the above description in the broadest scope. The preferred embodiments and examples should therefore merely be regarded as descriptive disclosure which is absolutely not limiting in any way.

The substances obtained are characterised by means of NMR spectroscopy. NMR spectra are recorded using the Bruker Avance 400 spectrometer from Bruker, Karlsruhe. The referencing takes place with external reference: TMS for ¹H and ¹³C spectra.

### EXAMPLES

### Example 1

### Synthesis of tributyl(methyl)ammonium octafluoropentoxybutanesulfonate

0.5 mol of octafluoropentanol are initially introduced with 50 g of 2-propanol at room temperature, and 0.5 mol of a 50% NaOH solution are subsequently added in portions. The mixture is heated to 80° C., and 0.5 mol of butanesultone is added dropwise over the course of one hour. The mixture is subsequently stirred at 80-90° C. for a further hour. The product sodium octafluoropentoxybutanesulfonate is recrystallised from methanol and is in the form of flakes with a mother-of-pearl shimmer.

For the salt exchange, the substance obtained from the first reaction is dissolved in water and reacted with an equivalent amount of tributyl(methyl)ammonium chloride, likewise dissolved in water, with addition of dichloromethane (1 ml/g of product). After phase separation, the aqueous phase is extracted three times with CH₂Cl₂. The product-containing, organic phase is washed 5 times with DI water. The solvent is removed at 80° C. in vacuo, and the product is obtained as a viscous, pale-yellow liquid.

1H NMR (400 MHz, DMSO-d6) δ=7.02 (tt, J=50.3, 5.7, 1H), 4.05 (t, J=14.9, 2H), 3.56 (t, J=5.8, 2H), 3.33-3.11 (m, 6H), 2.98 (s, 3H), 2.42 (t, J=7.1, 2H), 1.80-1.45 (m, 10H), 1.32 (h, J=7.3, 6H), 0.94 (t, J=7.4, 9H).

### Example 2

### Corrosion Experiments

The respective metal is introduced into a Petri dish (ø30 mm h=12 mm), and covered with about 1.5 ml of IL (ionic liquid) or 1.5 ml of IL plus 1 per cent by weight of the additive according to Example 1. The Petri dishes are subsequently heated to 150° C. in a drying cabinet and conditioned. The visual assessment is carried out after 24 h, 100 h and 200 h. In each case, one Petri dish per IL without metal is also assessed as blank value.

The following ionic liquids and their behaviour with and without additive of Example 1 on samples made from steel 100Cr6, copper, steel St 37-2, stainless steel 1.4301, bronze and aluminium are investigated:

### BMPL OTF,

### BMPL NTF,

### E3MPYR NON,

### BMMIM NTF.

### GLOSSARY

++ no corrosion tendency
+ minimal discoloration of the metal
− corrosion tendency
.−− significant corrosion tendency
−−− very strong corrosion tendency

### Values for BMPL OTF:

TABLE 1

| | Additive | | | |
|---|---|---|---|---|
| | according to | | | |
| | Example 1 | Corrosion | Corrosion | Corrosion |
| Metal | [% by weight] | after 24 h | after 100 h | after 200 h |
| 100Cr6 | 0 | ++ | — | X |
| 100Cr6 | 1 | ++ | ++ | + |
| Cu | 0 | — | — | X |
| Cu | 1 | ++ | ++ | ++ |
| St 37-2 | 0 | — | — | X |
| St 37-2 | 1 | ++ | ++ | ++ |
| 1.4301 | 0 | ++ | + | X |
| 1.4031 | 1 | ++ | ++ | ++ |
| Bronze | 0 | — | — | X |
| Bronze | 1 | ++ | ++ | + |
| Aluminium | 0 | ++ | ++ | X |
| Aluminium | 1 | ++ | ++ | ++ |

The entry X means that no measurement was carried out owing to the clear picture of damage.

### Values for BMPL NTF:

TABLE 2

| | Additive | | | |
|---|---|---|---|---|
| | according to | | | |
| | Example 1 | Corrosion | Corrosion | Corrosion |
| Metal | [% by weight] | after 24 h | after 100 h | after 200 h |
| 100Cr6 | 0 | ++ | ++ | X |
| 100Cr6 | 1 | ++ | ++ | + |
| Cu | 0 | + | — | X |
| Cu | 1 | ++ | ++ | — |
| St 37-2 | 0 | — | X | X |
| St 37-2 | 1 | ++ | ++ | ++ |
| 1.4301 | 0 | ++ | ++ | X |
| 1.4031 | 1 | ++ | ++ | ++ |
| Bronze | 0 | + | — | X |
| Bronze | 1 | + | + | + |
| Aluminium | 0 | ++ | ++ | X |
| Aluminium | 1 | ++ | ++ | ++ |

### Values for B3MPYR NON:

TABLE 3

| | Additive | | | |
|---|---|---|---|---|
| | according to | | | |
| | Example 1 | Corrosion | Corrosion | Corrosion |
| Metal | [% by weight] | after 24 h | after 100 h | after 200 h |
| 100Cr6 | 0 | ++ | ++ | X |
| 100Cr6 | 1 | ++ | ++ | ++ |
| Cu | 0 | ++ | ++ | X |
| Cu | 1 | + | + | + |
| St 37-2 | 0 | — | X | X |
| St 37-2 | 1 | ++ | ++ | ++ |
| 1.4301 | 0 | ++ | — | X |
| 1.4031 | 1 | ++ | ++ | ++ |
| Bronze | 0 | ++ | — | X |
| Bronze | 1 | + | + | — |
| Aluminium | 0 | ++ | ++ | X |
| Aluminium | 1 | ++ | ++ | ++ |

### Values for BMMIM NTF:

TABLE 4

| | Additive | | | |
|---|---|---|---|---|
| | according to | | | |
| | Example 1 | Corrosion | Corrosion | Corrosion |
| Metal | [% by weight] | after 24 h | after 100 h | after 200 h |
| 100Cr6 | 0 | ++ | + | X |
| 100Cr6 | 1 | ++ | ++ | ++ |
| Cu | 0 | + | — | X |
| Cu | 1 | ++ | ++ | + |
| St 37-2 | 0 | + | — | X |
| St 37-2 | 1 | ++ | ++ | ++ |
| 1.4301 | 0 | ++ | ++ | X |
| 1.4031 | 1 | ++ | ++ | ++ |
| Bronze | 0 | — | — | X |
| Bronze | 1 | ++ | + | + |
| Aluminium | 0 | ++ | ++ | X |
| Aluminium | 1 | ++ | ++ | ++ |

### Example 3

### Experimental Procedure with the Compound According to Example 1 in Accordance with VDMA 24570

This method is a corrosion test which allows corrosion to continue due to constant mechanical activation of the test specimen surface and allows quantitative classification of the corrosiveness of the respective test liquid by determination of weight loss.

A liquid sample acts on ground test specimen surfaces from various material groups which have been constantly mechanically activated by glass beads, at a test temperature of 120° C. After a test duration of 96 hours, the change in weight of the test specimens in g/cm² is determined and quoted as weight loss of the individual material groups.

Equipment: laboratory power supply LSP-1403, corrosion stand, magnetic stirrer plate with temperature sensor.

### Preparation:

The sample length and width is determined to an accuracy of 0.05 mm using a vernier caliper. The test area (F) in m² can be calculated from the measurement results:

F(m²)=sample length (mm)*sample width (mm)/1,000,000.

The samples should always be degreased by the same method (dip sample in cyclohexane, acetone and methanol for 5 minutes each) and may only subsequently be touched using cotton gloves.

The sample weight should be determined to an accuracy of 0.1 mg by means of the analytical balance.

### Measurement (Double Determination):

The metal samples are installed in the holder.

150 g+/−0.21 g of sample liquid is introduced into a beaker, and 550 g+/−5 g of glass beads are introduced.

The liquid level of the heating bath must be 10 mm above the liquid level of the test liquid.

The holder is installed in the corrosion stand, the thermometer is installed so that the measurement point is 20 mm above the base of the beaker, and the stirrer is set in operation with a speed n=220+/−5 min⁻¹ (supply voltage 18 V). The heating is set in operation, where the heating duration are 70 to 80 minutes until the target temperature of 120° C. is reached. The test temperature must be kept at 120° C.+/−1° C. The test duration including heating rate is 96 h.

After the measurement and cooling to 70° C., the stirrer is switched off, and the holder is removed from the apparatus. The sample holder is cooled to RT and subsequently disassembled. The samples are rinsed with suitable solvent, carefully dried and weighed.

### Evaluation:

The sample weight should be determined to an accuracy of 0.1 mg, and the weight loss based on the initial test area should be determined in g/m²:

$$weight loss [ g \text{/} {m}^{2} ] = \frac{\begin{matrix}m ( sample before measurement ) [ mg ] - \\ m ( sample after measurement ) [ mg ]\end{matrix}}{test area [ {m}^{2} ] * 1000}$$

The abrasion per year [mm/a] is determined as follows:

mm/a=((weight loss [g]/(8700 h/measurement time [h]))/δ[g/mm³])/area [mm²]

The following table shows the VDMA values obtained with and without additive according to Example 1 for the ionic liquid BMPL OTF:

| | Cu | Bronze | St 37-2 | 1.4301 |
|---|---|---|---|---|
| Abrasion in mm/a without additive | 1.878 | 2.69 | 0.283 | 0.093 |
| Abrasion in mm/a with additive | 0.025 | 0.004 | 0.023 | 0.028 |
| according to Example 1 | | | | |

It is clear from the abrasion values that, in particular, nonferrous metal corrosion can be reduced by addition of the additive according to Example 1.

1. Compounds of the formula I
[Kt]^{z+}z[SO₃—(CH₂)ₓ—O—CH₂—(CF₂—CF₂)_{y}—H]⁻ I
in which [Kt]^{z+} denotes an inorganic or organic cation, z denotes 1, 2, 3 or 4, y denotes 1, 2 or 3 and x denotes 3 or 4, where the compounds sodium nonafluoropropoxypropanesulfonate, sodium octafluoropentoxypropanesulfonate and sodium dodecafluoroheptoxypropanesulflonate are excluded.

2. Compounds according to claim 1 characterised in that the cation is an organic cation.

3. Compounds according to claim 1, characterised in that y denotes 2.

4. Compounds according to claim 1, characterised in that x denotes 4.

5. Compounds according to claim 1, characterised in that the cation [Kt]^{z+} is selected from
a sulfonium cation of the formula (1)
[(R^{o}₃S]⁺ (1),
where R^{o} in each case, independently of one another, denotes (R′″)₂N,
a linear or branched alkyl group having 1 to 8 C atoms,
an unsubstituted phenyl group or a phenyl group which is mono- or polysubstituted by R¹*, OR′, SR′, N(R′)₂, CN or halogen,
where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl,
R¹* in each case, independently of one another, denotes unfluorinated or partially fluorinated linear or branched alkyl group having 1 to 8 C atoms, saturated cycloalkyl group having 3 to 7 C atoms or unsubstituted or substituted phenyl and
R′″ in each case, independently of one another, denotes a linear or branched alkyl group having 1 to 6 C atoms;
an ammonium cation of the formula (2)
[NR₄] (2),
where
R in each case, independently of one another,
denotes H, OR′ or N(R′)₂, with the assumption that a maximum of one substituent R in formula (3) denotes OR′ or N(R′)₂,
denotes a linear or branched alkyl group having 1 to 20 C atoms,
denotes a linear or branched alkenyl group having 2 to 20 C atoms and at least one double bond,
denotes a linear or branched alkynyl group having 2 to 20 C atoms and at least one triple bond or
denotes a saturated, partially unsaturated, unsaturated or aromatic cyclic group having 3 to 7 C atoms, which may be mono- or polysubstituted by a linear or branched alkyl group having 1 to 6 C atoms,
where one or two R may be partially replaced by halogen, preferably —F and/or —Cl, and/or by —OR′, —CN, —N(R′)₂, —C(O)OR′, —C(O)R′, —C(O)N(R′)₂, —SO₂N(R′)₂, —C(O)X, —SR′, —S(O)R′, —SO₂R′ and where one or two carbon atoms of the radical R which are not adjacent and are not in the α-position may be replaced by atoms and/or atom groups selected from the group —O—, —S—, —S(O)—, —SO₂—, —SO₂O—, —C(O)—, —C(O)O—, —N⁺(R′)₂—, —P(O)R′O—, —C(O)NR′—, —SO₂NR′—, —OP(O)R′O—, —P(O)(N(R′)₂)NR′—, —P(R′)₂═N— or —P(O)R′—, where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl and
X denotes F, Cl, Br or I;
a phosphonium cation of the formula (3)
[P(R²)₄]⁺ (3),
where
R² in each case, independently of one another,
denotes H, OR′ or N(R′)₂,
denotes a linear or branched alkyl group having 1 to 20 C atoms,
denotes a linear or branched alkenyl group having 2 to 20 C atoms and at least one double bond,
denotes a linear or branched alkynyl group having 2 to 20 C atoms and at least one triple bond or
denotes a saturated, partially unsaturated, unsaturated or aromatic cyclic group having 3 to 7 C atoms, which may be mono- or polysubstituted by a linear or branched alkyl group having 1 to 6 C atoms,
where one or two R² may be partially replaced by halogen, preferably —F and/or —Cl, and/or by —OR′, —CN, —N(R′)₂, —C(O)OR′, —C(O)R′, —C(O)N(R′)₂, —SO₂N(R′)₂, —C(O)X, —SR′, —S(O)R′, —SO₂R′ and where one or two carbon atoms of the radical R² which are not adjacent and are not in the α-position may be replaced by atoms and/or atom groups selected from the group —O—, —S—, —S(O)—, —SO₂—, —SO₂O—, —C(O)—, —C(O)O—, —N⁺(R′)₂—, —P(O)R′O—, —C(O)NR′—, —SO₂NR′—, —OP(O)R′O—, —P(O)(N(R′)₂)NR′—, —P(R′)₂═N— or —P(O)R′—, where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl and
X denotes F, Cl, Br or I;
a thiouronium cation of the formula (4),
[C(NR³R⁴)(SR⁵)(NR⁶R⁷)]⁺ (4),
where
R³ to R⁷ each, independently of one another,
denote H, where H is excluded for R⁵,
denotes a linear or branched alkyl group having 1 to 20 C atoms,
denotes a linear or branched alkenyl group having 2 to 20 C atoms and at least one double bond,
denotes a linear or branched alkynyl group having 2 to 20 C atoms and at least one triple bond or
denotes a saturated, partially unsaturated, unsaturated or aromatic cyclic group having 3 to 7 C atoms, which may be mono- or polysubstituted by a linear or branched alkyl group having 1 to 6 C atoms,
where one or two of the substituents R³ to R⁷ may be partially replaced by halogen, preferably —F and/or —Cl, and/or by —OR′, —CN, —N(R′)₂, —C(O)OR′, —C(O)R′, —C(O)N(R′)₂, —SO₂N(R′)₂, —C(O)X, —SR′, —S(O)R′, —SO₂R′ and where one or two carbon atoms of the radicals R³ to R⁷ which are not adjacent and are not in the α-position may be replaced by atoms and/or atom groups selected from the group —O, S, S(O)—, —SO₂—, —SO₂O—, —C(O)—, —C(O)O—, —N⁺(R′)₂—, —P(O)R′O—, —C(O)NR′—, —SO₂NR′—, —OP(O)R′O—, —P(O)(N(R′)₂)NR′—, —P(R′)₂═N— or —P(O)R′—, where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl and
X denotes F, Cl, Br or I;
a guanidinium cation of the formula (5)
[C(NR⁸R⁹)(NR¹⁰R¹¹)(NR¹²R¹³)]⁺ (5),
where
R⁸ to R¹³ each, independently of one another,
H, —CN, N(R′)₂, —OR′,
denotes a linear or branched alkyl group having 1 to 20 C atoms,
denotes a linear or branched alkenyl group having 2 to 20 C atoms and at least one double bond,
denotes a linear or branched alkynyl group having 2 to 20 C atoms and at least one triple bond or
denotes a saturated, partially unsaturated, unsaturated or aromatic cyclic group having 3 to 7 C atoms, which may be mono- or polysubstituted by a linear or branched alkyl group having 1 to 6 C atoms,
where one or two of the substituents R⁸ to R¹³ may be partially replaced by halogen, preferably —F and/or —Cl, and/or by —OR′, —CN, —N(R′)₂, —C(O)OR′, —C(O)R′, —C(O)N(R′)₂, —SO₂N(R′)₂, —C(O)X, —SR′, —S(O)R′, —SO₂R′ and where one or two carbon atoms of the radicals R⁸ to R¹³ which are not adjacent and are not in the α-position may be replaced by atoms and/or atom groups selected from the group —O—, —S—, —S(O)—, —SO₂—, —SO₂O—, —C(O)—, —C(O)O—, —N⁺(R′)₂—, —P(O)R′O—, —C(O)NR′—, —SO₂NR′—, —OP(O)R′O—, —P(O)(N(R′)₂)NR′—, —P(R′)₂═N— or —P(O)R′—, where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl and
X denotes F, Cl, Br or I;
a heterocyclic cation of the formula (6),
[HetN]^{z+} (6),
where
HetN^{z+} denotes a heterocyclic cation selected from the group 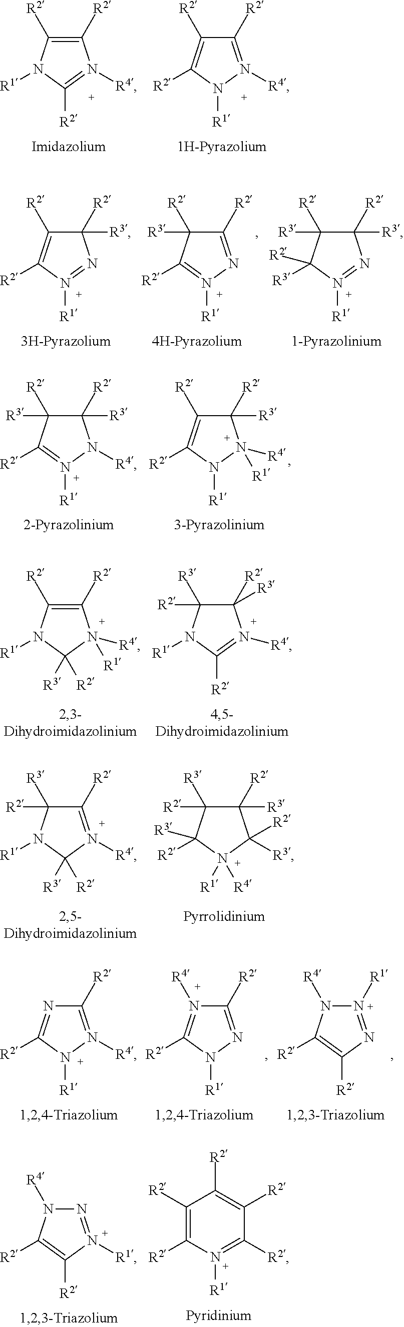 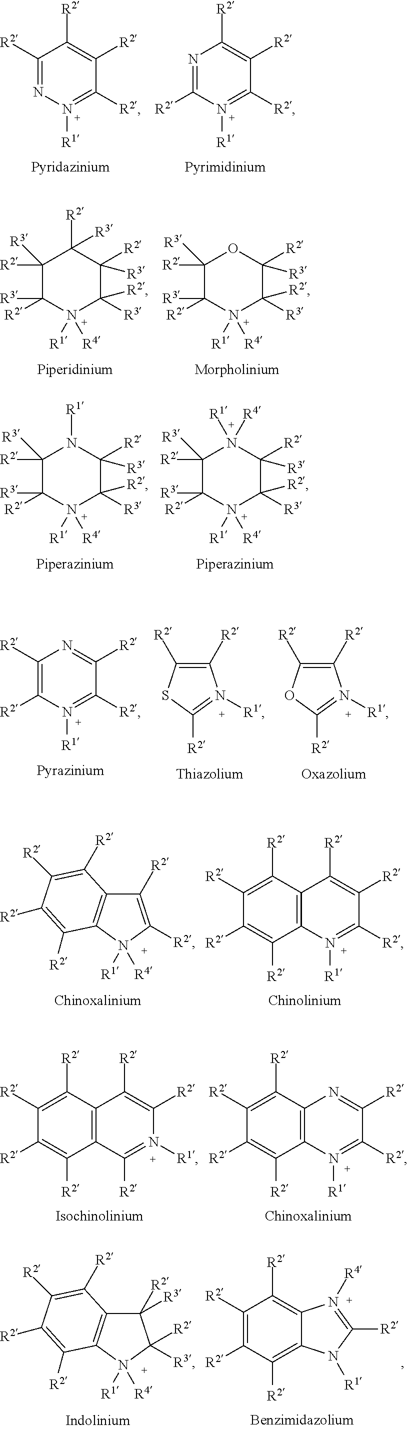 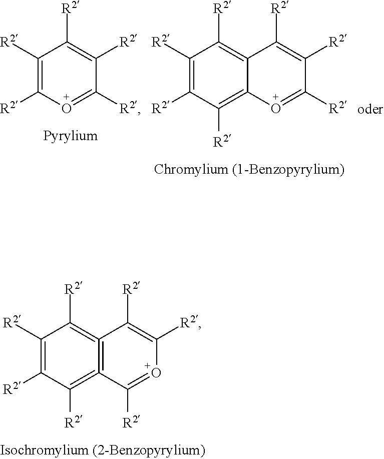
where the substituents
R¹′ to R⁴′ each, independently of one another, denote
H,
linear or branched alkyl group having 1 to 20 C atoms,
linear or branched alkenyl group having 2 to 20 C atoms and at least one double bond,
linear or branched alkynyl group having 2 to 20 C atoms and at least one triple bond,
cycloalkyl group having 3 to 7 C atoms, which may be mono- or polysubstituted by a linear or branched alkyl group having 1 to 6 C atoms,
unsubstituted phenyl or phenyl which is mono- or polysubstituted by a linear or branched alkyl group having 1 to 6 C atoms,
heteroaryl, heteroaryl-C₁-C₆-alkyl or aryl-C₁-C₆-alkyl
and
R^{2′} may additionally denote F, Cl, Br, I, —CN, —OR′, —N(R′)₂, —P(O)(R′)₂, —P(O)(OR′)₂, —P(O)(N(R′)₂)₂, —C(O)R′, —C(O)OR′, —C(O)X, —C(O)N(R′)₂, —SO₂N(R′)₂, —SO₂OH, —SO₂X, —SR′, —S(O)R′, and/or —SO₂R′, with the assumption that the substituents R¹′, R³′, R⁴′ then each, independently of one another, denote H and/or a linear or branched alkyl group having 1 to 20 C atoms and/or a linear or branched alkenyl group having 2 to 20 C atoms and at least one double bond,
where the substituents R^{1′}, R^{2′}, R^{3′} and/or R^{4′} together may also form a ring system,
where one or more substituents R^{1′} to R^{4′} may also be partially replaced by halogen, preferably F and/or Cl, and/or by —OR′, N(R′)₂, —CN, —C(O)OR′, —C(O)R′, —C(O)N(R′)₂, —SO₂N(R′)₂, —C(O)X, —SR′, —S(O)R′, —SO₂R′, but where R^{1′} and R^{4′} cannot simultaneously be fully substituted by halogen and where one or two carbon atoms of the radicals R^{1′} to R^{4′} which are not adjacent and are not in the α-position may be replaced by atoms and/or atom groups selected from the group —O, S, S(O)—, —SO₂—, —SO₂O—, —C(O)—, —C(O)O—, —N⁺(R′)₂—, —P(O)R′O—, —C(O)NR′—, —SO₂NR′—, —OP(O)R′O—, —P(O)(N(R′)₂)NR′—, —P(R′)₂═N— or —P(O)R′—, where
R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl and
X denotes F, Cl, Br or I;
an iodonium cation of the formula (7)
Ar-I⁺-Ar (7),
where
Ar in each case, independently of one another, denotes an aryl group having 6 to 30 C atoms, which may be unsubstituted or substituted by at least one linear or branched alkenyl group having 2 to 20 C atoms and at least one double bond, a linear or branched alkynyl group having 2 to 20 C atoms and at least one triple bond and/or by R¹*, SR′^{′}, N(R′)₂, CN and/or halogen, where R′ in each case, independently of one another, denotes H, unfluorinated or partially fluorinated linear or branched alkyl group having 1 to 8 C atoms, saturated cycloalkyl group having 3 to 7 C atoms or unsubstituted or substituted phenyl and
R¹* in each case, independently of one another, denotes unfluorinated or partially fluorinated linear or branched alkyl group having 1 to 8 C atoms, saturated cycloalkyl group having 3 to 7 C atoms or unsubstituted or substituted phenyl
and
halogen denotes F, Cl, Br or I.

6. Process for the preparation of compounds of the formula I according to claim 1 and where x in formula I denotes 4, characterised in that an alcohol of the formula II
H—(CF₂—CF₂)_{y}—CH₂—OH II,
in which y denotes 1, 2 or 3,
in a suitable solvent is treated with sodium hydroxide solution and subsequently with 1,4-butanesultone, and the sodium salt obtained is optionally subjected to salt exchange in a metathesis reaction with compounds of the formula III,
KtA III,
to give the corresponding compound of the formula I,
where Kt denotes an inorganic or organic cation, where Na⁺ is excluded, and
A is selected from the group of the anions Cl⁻, Br⁻, I⁻, [HF₂]⁻, [R₁SO₃]⁻, [R₂COO]⁻, [R₂SO₃]⁻, [R₁OSO₃]⁻, [PF₆]⁻, [BF₄]⁻, [SO₄]²⁻, [HSO₄]¹⁻, [NO₃]⁻, [(R₂)₂P(O)O]⁻, [R₂P(O)O₂]²⁻, [(R₁O)₂P(O)O]⁻, ([R₁O)P(O)O₂]²⁻, [(R₁O)R₁P(O)O]⁻, [HOCO₂]⁻ or [CO₃]²⁻, with the assumption that the anions [SO₄]²⁻ and [CO₃]²⁻ are used for the preparation of compounds of the formula I containing other alkali-metal cations and
where
R₁ in each case, independently of one another, denotes a linear or branched alkyl group having 1 to 12 C atoms and
R₂ in each case, independently of one another, denotes a linear or branched perfluorinated alkyl group having 1 to 12 C atoms and
where the electroneutrality of the salt KtA is observed.

7. Process for the preparation of compounds of the formula I according to claim 1 and where x in formula I denotes 3, characterised in that an alcohol of the formula II
H—(CF₂—CF₂)_{y}—CH₂—OH II,
in which y denotes 1, 2 or 3,
is reacted with allyl chloride and sodium hydroxide solution in a suitable solvent and in the presence of a phase-transfer catalyst, the allyl ether of the formula IV,
H—(CF₂—CF₂)_{y}—O—CH₂—CH═CH₂ IV,
in which y denotes 1, 2 or 3,
formed as an intermediate is reacted with an aqueous NaHSO₃/Na₂SO₃ solution at a pH of 7 to 8 in the presence of a solubiliser, and the sodium salt formed is subjected to salt exchange in a metathesis reaction with compounds of the formula III,
KtA III,
to give the corresponding compound of the formula I,
where Kt denotes an inorganic or organic cation, where Na⁺ is excluded, and
A is selected from the group of the anions Cl⁻, Br⁻, I⁻, [HF₂]⁻, [R₁SO₃]⁻, [R₂COO]⁻, [R₂SO₃]⁻, [R₁OSO₃]⁻, [PF₆]⁻, [BF₄]⁻, [SO₄]²⁻, [HSO₄]¹⁻, [NO₃]⁻, [(R₂)₂P(O)O]⁻, [R₂P(O)O₂]²⁻, [(R₁O)₂P(O)O]⁻, [(R₁O)P(O)O₂]²⁻, [(R₁O)R₁P(O)O]⁻, [HOCO₂]⁻ or [CO₃]²⁻, with the assumption that the anions [SO₄]²⁻ and [CO₃]²⁻ are used for the preparation of compounds of the formula I containing other alkali-metal cations and
where
R₁ in each case, independently of one another, denotes a linear or branched alkyl group having 1 to 12 C atoms and
R₂ in each case, independently of one another, denotes a linear or branched perfluorinated alkyl group having 1 to 12 C atoms and
where the electroneutrality of the salt KtA is observed.

8. Composition comprising at least one compound of the formula I according to claim 1.

9. Composition according to claim 8, characterised in that it is a lubricant or a lubricating oil.

10. Composition according to claim 8, characterised in that, besides the compound of the formula I according to one or more of claims 1 to 5, at least one further ionic compound is present which does not conform to the formula I and is not a phosphate or alkylsulfate.

11. A method of inhibiting corrosion of metal surface comprising applying a compound of the formula I according to claim 1 as an anticorrosion additive.

12. A method according to claim 11, wherein said compound is combined together with ionic compounds which do not conform to the formula I and are not phosphates or alkylsulfates.

13. A method according to claim 11 for inhibition of the corrosion of nonferrous metals.

14. A method according to claim 12, characterised in that the ionic compound is an ionic liquid.

15. Use of compounds of the formula I according to claim 2, where the cations conform to the formula (1), (4) or (7) or the formulae for pyrylium, 1-benzopyrylium or 2-benzopyrylium, as cationic polymerisation initiator, photopolymerisation initiator or as photo-acid generator.

16. Curable composition comprising at least one compound of the formula I according to claim 2, where the cations conform to the formula (1), (4) or (7) or the formulae for pyrylium, 1-benzopyrylium or 2-benzopyrylium, and at least one polymerisable compound.
